(19) United States
(12) Patent Application Publication
(10) Pub. No.: US 2022/0340949 A1
(43) Pub. Date: Oct. 27, 2022

(54) Methods of Isoprenoid Synthesis Using a Genetically Engineered Hydrocarbonoclastic Organism in a Biofilm Bioreactor

(71) Applicant: Capra Biosciences, Inc., Alexandria, VA (US)

(72) Inventors: Andrew P. Magyar, Acton, MA (US); Elizabeth Onderko, Alexandria, VA (US)

(21) Appl. No.: 17/722,182

(22) Filed: Apr. 15, 2022

Related U.S. Application Data
(60) Provisional application No. 63/175,858, filed on Apr. 16, 2021.

(51) Int. Cl. C12P 23/00; C12N 1/20; C12N 15/52; C12N 15/74; C12M 1/12; C12M 1/00; C12P 7/62; C12P 5/00; C12P 5/02; A61K 8/67; A61K 8/31; A61Q 19/00

(52) U.S. Cl. CPC C12P 23/00; C12N 1/20; C12N 15/52; C12N 15/74; C12M 25/18; C12M 29/06; C12M 47/10; C12P 7/62; C12Y 113/11063; C12Y 101/013; C12P 5/007; C12P 5/026; A61K 8/671; A61K 8/31; A61Q 19/00; C12N 2800/101; A61K 2800/85

(57) ABSTRACT

Described herein are genetically-engineered organisms comprising synthetic operons for the production of isoprenoids, carotenoids, and retinoids, optimized for use in a hydrocarbonoclastic organism, and methods for the synthesis and extraction of isoprenoids in a biofilm bioreactor comprising the genetically-engineered organisms.

### RELATED APPLICATIONS

This application claims the benefit under 35 USC 119(e) of U.S. Provisional Application No. 63/175,858, filed on Apr. 16, 2021, which is incorporated herein by reference in its entirety.

### INCORPORATION BY REFERENCE OF MATERIAL IN ASCII TEXT FILE

The instant application contains a Sequence Listing which has been submitted electronically in ASCII format and is hereby incorporated by reference in its entirety. Said ASCII copy, created on Apr. 14, 2022, is named 0412_0001US1_SL.txt and is 79,417 bytes in size.

### BACKGROUND OF THE INVENTION

Isoprenoids or terpenoids are a class of molecules derived from the 5-carbon compound isoprene. In nature, this encompasses molecules responsible for the flavors of many spices, and pigment molecules such as carotenoids. Isoprenoids also serve as precursors for the synthesis of sterols such as cholesterol and steroids. While many of these molecules are found in nature, and can be synthesized biologically, commercial manufacture of compounds such as retinol, a common cosmetic ingredient, involve organic synthesis from petrochemical-derived precursors.

Many isoprenoids can also be derived from plants, where they naturally occur—but the concentrations of these compounds are quite low (mg/kg), leading to inefficient production, costly products, and significant waste. Fermentation has attracted great interest as an alternative approach for manufacturing isoprenoids, as described by Keasling et al., in U.S. Pat. No. 7,172,886. Production of carotenoids have been explored in oleaginous fungi and yeast (U.S. Pat. No. 8,288,149 B2). The low aqueous solubility of many isoprenoids, however, limits the commercial viability of their production through traditional fermentation.

To overcome these solubility limitations as well as toxicity due to accumulated product, the use of an overlay of a nonpolar organic solvent such as hexane or dodecane can be applied to the fermentation broth to extract hydrophobic products into the second phase, a design known as a two phase partitioning bioreactor (Daugulis 1997; Malinowski 2001). In particular, solvent overlays have been used for the commercial production of the sesquiterpene farnesene, in a fed batch process (U.S. Pat. No. 10,106,822 B2). A similar approach has been employed for the fermentative synthesis of retinoids (U.S. Pat. No. 9,834,794 B2, Jang et al., 2011; Sun et al., 2019) in a two-phase system with in situ extraction.

Traditional two phase extraction and partitioning bioreactor approaches are limited because the organisms are typically not tolerant of the solvents and so the solvent is not in direct contact with the organisms. Hydrophobic products must diffuse through an aqueous phase in which they have low solubility before they are extracted from the reactor, which can limit the reactor productivity. Faster and more efficient methods of synthesizing hydrophobic organic molecules such as isoprenoids and retinoids are needed.

### SUMMARY OF THE INVENTION

The present invention encompasses compositions, methods, and apparatus for producing isoprenoids, carotenoids and retinoids using hydrocarbonoclastic organisms in a biofilm bioreactor. A biofilm bioreactor, (for example as described in U.S. Application 62/978,428/WO 2021/168039/US 2021/0253990, the teachings of which are incorporated herein by reference), that can be adapted for use with in situ solvent extraction can overcome the limitations of producing bioorganic, hydrophobic molecules as described above, but would require a solvent-tolerant biofilm forming organism Specifically, the engineering of a hydrocarbonoclastic organism (also known as hydrocarbon degrading bacteria) with a pathway to produce isoprenoids and retinoids can enable more efficient methods of biological isoprenoid and retinoid synthesis through direct extraction using organic solvents, for example, using a biofilm or biofilm reactor.

Encompassed by the present invention are methods of synthesis comprising the use of genetically-engineered hydrocarbonoclastic organisms selected from species of prokaryotes or archaea which can degrade and utilize hydrocarbon compounds as a source of carbon and energy. As described herein these hydrocarbonoclastic organisms, are used in methods to produce (biosynthesize) a class of compounds called isoprenoids or terpenoids (terpenoids/isoprenoids are organic compounds derived from the 5-carbon compound-isoprene and isoprene polymers, terpenes). Degrading and utilizing hydrocarbons are a characteristic of hydrocarbonoclastic organisms such as Marinobacter spp. (Gauthier, 1992; Handley, 2013) or Pseudomonas spp. (Isken, 1998).

Specifically encompassed by the present invention are hydrocarbonoclastic microorganisms that are genetically-engineered for increased biological activity relative to its wild-type organism to synthesize/produce isoprenoids, carotenoids, or retinoids (e.g., wherein the product molecule/compound is e.g., retinal or retinol) in high yield in a biofilm or biofilm bioreactor.

The hydrocarbononoclastic microorganisms suitable for use in the present invention have, inter alia, two important characteristics: the ability to form a stable biofilm (e.g., in a biofilm reactor) and a tolerance to hydrophobic organic solvents. Such microorganisms include, for example, Marinobacter species, and Pseudomonas species. More specifically, encompassed by the present invention are biofilm-forming hydrocarbonoclastic microorganisms, such as Marinobacter spp., and in particular Marinobacter atlanticus, that are capable of forming biofilms and have tolerance to hydrophobic organic solvents. Such hydrocarbononoclastic organisms are genetically-engineered to contain one, or more nucleic acid or amino acid sequence variations/mutations in one, or more (e.g., a plurality of) genes that make up the mevalonate and/or carotene synthetic pathway. In particular, the present invention provides nucleic acid (DNA) sequences (SEQ ID NOS: 1-30 as shown in FIGS. 1-30) that encode for the genetically-engineered operons and/or genes in the mevalonate, beta-carotene, and retinol pathways and are engineered, for example, through codon harmonization for expression in Marinobacter spp.

The operon is responsible for gene expression and protein synthesis in prokaryotes. An operon, as described herein, is a grouping of (or a region of) one, or more related genes/gene sequences which are expressed to produce one, or more biologically active enzymes/proteins. The operon comprises one, or more, gene sequences encoding the desired proteins, a promoter sequence and an operator sequence (the operator sequence can be located within the promoter sequence or as a separate sequence). The operon is responsible for the transcription of DNA into messenger RNA (mRNA) which is then translated into the desired protein(s) or enzyme product in the prokaryote.

As described herein, the present invention encompasses variant operons/genes that encode enzymes/proteins having biological activity that differs from its counterpart wild-type (non-altered) operons/genes. One example of increased biological activity of the variant operon/gene over its wild-type counterpart is to increase the yield of the desired product such as the retinoid compound. Another biological activity described herein is the ability/capability to form a more stable biofilm, for example in a bioreactor. Another biological activity described herein is increased stability to organic solvents. One example of increased biological activity of the variant operon/gene over its wild-type counterpart is to allow the expression of the necessary genes and subsequent enzymes in a hydrocarbonoclastic/oleaginous biofilm forming organism.

For example, in some embodiments of the present invention, the variant genes encode genetically-engineered promoter sequences to increase expression of the desired enzymes, thus resulting in increased synthesis, yield or stability of the desired retinoid compound. In another example, the specific genes comprising an operon can be rearranged resulting in a variant operon that differs in biological activity from the wild-type operon, again resulting in increased synthesis, yield or stability of the desired retinoid product.

More specifically encompassed by the present invention, is a genetically engineered (also referred to herein as genetically modified or altered) hydrocarbonoclastic microorganism wherein the operon genes encoding enzymes required for mevalonate production pathway have been optimized by codon harmonization (also referred to herein as synthetic genes), wherein the pathway is designed to route the native acetyl-CoA pool of Marinobacter spp. or a similar hydrocarbonoclastic organism to the production of isoprenoids such as retinal or retinol. Such microorganisms can be further modified to include variant genes/operons of the beta-carotene synthetic pathway designed to route beta-carotene to the production of retinal, retinoate, retinol or retinyl esters.

As described herein, these enzymes (also referred to herein as variant proteins or synthetic enzymes) from these pathways will be engineered to improve performance, that is, the nucleotide sequences that encode for these modified enzyme sequences will be optimized (e.g., by codon harmonization, mutations, insertions or alterations resulting in the variant enzymes differing in sequence and biological activity from their wild-type/naturally-occurring counterpart enzyme) to alter/modify (typically increase) the biological/catalytic activity of the enzymes as compared to (i.e., relative to) enzymatic activity encoded by the operon genes in the wild-type (unmodified) microorganism. Methods of genetically engineering the microorganisms are described herein and methods of evaluating enzymatic/biological activity of proteins are also described herein and are known to those of skill in the art.

The genetically engineered microorganisms of the present invention include/comprise the specific arrangement of the plurality of genes for retinal or retinol synthesis into an operon in which the genes are arranged in a manner such that enzyme expression is optimized for highest yield product synthesis.

Specifically described herein are organisms comprising variant genes in the mevalonate synthetic pathway (also referred to herein as the MVA pathway as in FIG. 31), encoding enzymes that convert acetyl-CoA into isopentenyl pyrophosphate (IPP), which is the building block of all isoprenoids (U.S. Pat. No. 7,172,866 B2, the teachings of which are incorporated herein by reference in their entirety). Such variant genes include the nucleic acid Sequences 5, 6, 7, 8, 9, 10 or 15, (SEQ ID NOS: 5, 6, 7, 8, 9, 10 or 15) or sequences comprising about 80, 85, 90, 95, 96, 97, 98 or 99% sequence identity to Sequences 5, 6, 7, 8, 9, 10 or 15.

In some embodiments, the organism is also genetically-engineered with one, or more, additional variant genes introduced into the organism wherein such genes make up the beta-carotene pathway, which encodes enzymes that convert IPP to beta-carotene. (See FIG. 32). Such variant genes include the nucleic acid Sequences 11, 12, 13, 14, 15, 17, 18, 19, 20, 21, 27, 28 or 29 (SEQ ID NOS: 11, 12, 13, 14, 15, 17, 18, 19, 20, 21, 27, 28 or 29), or sequences comprising about 80, 85, 90, 95, 96, 97, 98, or 99% sequence identity to Sequences 11, 12, 13, 14, 15, 17, 18, 19, 20, 21, 27, 28 or 29.

In one embodiment the genetically-engineered organism comprises introduction of a variant blh gene encoding the 15,15′-dioxygenase, (Sequence 16) wherein the introduction of the variant blh gene results in the production of retinal and/or retinol.

Also encompassed by the present invention is a variant human retinol dehydrogenase 12 (RDH12) gene encoding the retinol dehydrogenase comprising Sequence 30, and its encoded protein comprising Sequence 30. The retinol dehydrogenase gene (RDH12) can comprise a nucleic acid sequence selected from the group consisting of: Sequence 17, Sequence 18; or Sequence 20 (SEQ ID NOS: 17, 18 or 30), or a sequence comprising about 80, 85, 90, 95, 96, 96, 98, or 99% sequence identity to Sequences 18, 19 or 20. The encoded RDH12 protein can also encompass sequences comprising about 80, 85, 90, 95, 96, 97, 98, or 99% sequence identity to SEQ ID NO: 30, wherein the protein has aldehyde dehydrogenase biological activity comparable to the variant RDH12 activity. Further encompassed by the present invention are genetically-engineered organisms wherein the organism comprises introduction of a variant RDH12 gene (SEQ ID NOS: 17, 18 or 20) expressing a variant retinol dehydrogenase 12 (RDH12) SEQ ID NO: 30), wherein the introduction of the variant RDH12 gene results in the conversion of retinal to retinol.

Also encompassed is a genetically-engineered organism, wherein the organism comprises introduction of a variant ybbO gene (SEQ ID NOS:19 or 21), wherein the introduction of the variant ybbO gene results in the conversion of retinal to retinol.

Also encompassed herein are genetically-engineered organisms comprising the variant operon sequences described herein. For example, the organisms of the present invention can comprise a variant operon of the upper mevalonate pathway comprising Sequence 1(SEQ ID NO:1) and/or a variant operon of the lower mevalonate pathway comprising Sequence 2 (SEQ ID NO:2).

The genetically-engineered organisms of the present invention can further comprise variant operon sequences of the beta-carotene pathway, wherein a variant operon sequence is selected from the group of sequences consisting of: Sequence 3; Sequence 4; Sequence 22; Sequence 23; Sequence 24 or Sequence 25 (SEQ ID NOS: 3, 4, 22, 23, 24 or 25).

One particular embodiment of the present invention comprises the genetically-engineered organism, wherein the variant mevalonate pathway gene(s) comprise the variant operons of SEQ ID NO: 1 and SEQ ID NO: 2, and the variant carotene pathway gene(s) comprise the variant operons of SEQ ID NO: 3 and SEQ ID NO: 4. Another particular embodiment comprises the genetically-engineered organism, wherein the variant mevalonate pathway gene(s) comprise the variant operons of SEQ ID NO: 1 and SEQ ID NO: 2, and the variant carotene pathway gene(s) comprise the variant operons of SEQ ID NO:22 and SEQ ID NO: 26.

All nucleic acid sequences and amino acid sequences described herein include sequences with sequence identities of about 80, 85, 90, 95, 96, 97, 98, or 99% sequence identity to the described sequences. Such sequences will have comparable biological activity (essentially the same within a few measures of activity) as the described sequence when evaluated using standard techniques.

In some embodiments of the present invention, these genes/operons are introduced into an expression vector, such as a plasmid, suitable/compatible for expression of the genes in a competent host cell. Specifically, the host as described herein, is a hydrocarbonoclastic microorganism, specifically a Marinobacter species organism, and more specifically a Marinobacter atlanticus microorganism. After introduction of the expression vector comprising the variant genes of the present invention, under suitable conditions well known to those of skill in the art, the variant genes are incorporated/inserted into the genome of the host organism for expression. Techniques of genetic transfer into cells are known to those of skill in the art. Also encompassed by the present invention are host cells comprising the vectors or plasmids described herein.

In some embodiments, the hydrocarbonoclastic organism produces isoprenoids, carotenoids, or retinoids from aromatic or aliphatic molecules. In yet other embodiments the hydrocarbonoclastic organism produces isoprenoids, carotenoids, or retinoids from short chain fatty acids. In some of these embodiments, the short chain fatty acid is lactate from dairy waste.

The present invention further encompasses a biofilm comprising a genetically-engineered hydrocarbonoclastic microorganism as described herein, and a biofilm bioreactor, as described in U.S. patent application 62/978,428, now published as WO/2021/168039/US 2012/0253990, the teachings of which are incorporated herein by reference in their entirety, which contains a biofilm of the genetically-modified, isoprenoid-producing organism on a particulate support with an integrated system for product extraction with a hydrophobic solvent. The biofilm bioreactor can comprise, for example, a solid phase, support or matrix such as a packed bed, wherein the solid phase comprises particles or beads suitable for supporting the biofilm of hydrocarbonoclastic microorganisms described herein. Such a bioreactor is as shown in FIG. 33. which has an inlet for the introduction of media such as culture media that sustains the growth of the organisms of the biofilm and maintains the organisms producing the variant enzymes as described herein. The inlet is also suitable for the introduction of feedstock to supply the necessary factors for production of the desired end product (e.g., the isoprenoid of interest). A second inlet can be incorporated into the bioreactor for the introduction of an extraction solution e.g., to elute/harvest/obtain the desired product. For example, the extraction solution can be a non-polar solvent suitable for extracting the desired isoprenoid product with minimal alteration/disruption of the chemical structure of the product (i.e., partial or full destruction of the product).

As described herein, in some embodiments of the present invention, the bioreactor contains a mixer or nozzle to allow encapsulation of the extracted product to stabilize the end product and prevent or minimize degradation or oxidation.

Also encompassed by the present invention are methods for the production/synthesis of isoprenoids, carotenoids, and retinoids using a genetically engineered hydrocarbonoclastic organism such as Marinobacter species, or Pseudomonas species as described herein, in a biofilm or biofilm bioreactor. Specifically encompassed by the methods described herein is the production of the isoprenoids beta-carotene, retinal, retinol or squalane. Importantly, these methods comprise the use of organic solvents (e.g., non-polar solvents) to extract the isoprenoids without significant, or substantial, degradation of the isoprenoid product, resulting in higher yield, synthesis and/or stability of the desired product. For example, the synthetic biofilm and biofilm bioreactor described herein, and methods of synthesizing isoprenoids and retinoids can comprise the use of hexanes, dodecane, or oleic acid as an extraction solvent. of the desired product can be determined by techniques known to those of skill in the art.

In some embodiments, the extraction solvent specifically contains an anti-oxidant or an encapsulant to prevent oxidation or degradation of the product. For example, the extraction solvent can include a molecule such as cyclodextrin to stabilize the product molecule. In other embodiments, lipid molecules dispersed in solvent microdroplets are used to simultaneously extract the product and encapsulate the product in a liposome.

In a specific embodiment of the present invention, the method encompasses the production/synthesis of an isoprenoid used as an ingredient or component in the formulation of a cosmetic product, wherein the cosmetic ingredient is substantially free of contaminants that can be found in the isoprenoid produced by conventional methods. For example, the product retinol is often used in cosmetic creams or ointments manufactured for human use, so the purity of the retinol is extremely important. Evaluation of the purity, or degree of contamination, of lack of contamination, of the end product can be performed by methods known to those of skill in the art. Specifically, the product of the methods described herein is a cosmetic ingredient suitable for veterinary use or human use (e.g., retinol in a cosmetic facial cream) and the extraction solvent of the method is a component or a suitable additional ingredient of the cosmetic preparation/formulation. For example, the cosmetic ingredient can be an emollient, and in one embodiment the emollient is squalane.

Thus, as a result of the invention and its embodiments described herein, an improved, cost-effective method for the manufacture of isoprenoids, carotenoids, and retinoids is now available.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention.

The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. The patent or application file contains at least one drawing executed in color. Copies of this patent or patent application publication with color drawings(s) will be provided by the Office upon request and payment of the necessary fee. FIGS. 1-30 show the operon genes of the mevalonate and beta-carotene pathway optimized for isoprenoid synthesis in Marinobacter atlanticus.

FIG. 1. SEQUENCE 1 (SEQ ID NO:1): MVA1 (mvaE→mvaS). An operon containing the upper mevalonate pathway with codon harmonized versions of the mvaE gene and mvaS gene. These genes encode for the expression of acetyl-CoA acetyltransferase and HMG-CoA synthase, respectively. There is −1 spacing between genes in the operon.

FIG. 2. SEQUENCE 2 (SEQ ID NO:2): MVA2 (ldi→mvaK2→mvaD→mvaK1). An operon containing the lower mevalonate pathway and one enzyme from the beta carotene pathway with codon harmonized versions of the idi gene, mvaK2 gene, mvaD gene, and mvaK1 gene. These genes encode for the expression of isopentenyl-PP isomerase, phosphomevalonate kinase, mevalolonate-5 pyrophosphate decarboxylase, and mevalonate kinase, respectively. There is −1 spacing between genes in the operon.

FIG. 3. SEQUENCE 3 (SEQ ID NO:3): CRT1.1 (crtE→blh→crtY). An operon containing two genes in the beta carotene pathway and the gene for conversion of beta carotene to retinol, with codon harmonized versions of the crtE, blh, and crtY genes. These genes encode for the expression of GGPP synthase, 15,15′-dioxygenase, and lycopene cyclase.

FIG. 4. SEQUENCE 4 (SEQ ID NO:4): CRT2.1 (crtl→crtB→ispA). An operon containing three of the genes in the beta carotene pathway, with codon harmonized versions of the crtI, crtB, and ispA genes. These genes encode for the expression of phytoene desaturase, phytoene synthase, and farnesyl diphosphate synthase.

FIG. 5. SEQUENCE 5 (SEQ ID NO:5): mvaE. A codon harmonized version of mvaE, originating from Enterococcus faecalis and encoding for expression of acetyl-CoA transferase.

FIG. 6. SEQUENCE 6 (SEQ ID NO:6): mvaS. A codon harmonized version of mvaS, originating from Enterococcus faecalis and encoding for expression of HMG-CoA synthase.

FIG. 7. SEQUENCE 7 (SEQ ID NO:7): mvaK1. A codon harmonized version of mvaK1, originating from Streptococcus pneumoniae ATCC 6314 and encoding for expression of mevalonate kinase.

FIG. 8. SEQUENCE 8 (SEQ ID NO:8): mvaK2. A codon harmonized version of mvaK2, originating from Streptococcus pneumoniae ATCC 6314 and encoding for expression of phosphomevalonate kinase.

FIG. 9. SEQUENCE 9 (SEQ ID NO:9): mvaD. A codon harmonized version of mvaD, originating from Streptococcus pneumoniae ATCC 6314 and encoding for expression of mevalonate-5-pyrophosphate decarboxylase.

FIG. 10. SEQUENCE 10 (SEQ ID NO:10): idi. A codon harmonized version of mvaD, originating from Escherichia coli str. K-12 substr. W3110 and encoding for expression of isopentenyl-PP isomerase.

FIG. 11. SEQUENCE 11 (SEQ ID NO:11): crtE. A codon harmonized version of crtE, originating from Pantoea agglomerans KCCM 40420 and encoding for expression of GGPP synthase.

FIG. 12. SEQUENCE 12 (SEQ ID NO:12): crtB. A codon harmonized version of crtB, originating from Pantoea agglomerans KCCM 40420 and encoding for expression of phytoene synthase.

FIG. 13. SEQUENCE 13 (SEQ ID NO:13): crtI. A codon harmonized version of crtI, originating from Pantoea agglomerans KCCM 40420 and encoding for expression of phytoene desaturase.

FIG. 14. SEQUENCE 14 (SEQ ID NO: 14): crtY. A codon harmonized version of crtY, originating from Pantoea agglomerans KCCM 40420 and encoding for expression of lycopene cyclase.

FIG. 15. SEQUENCE 15 (SEQ ID NO:15): ispA. A codon harmonized version of ispA, originating from Escherichia coli str. K-12 substr. W3110 and encoding for expression of farnesyl diphosphate synthase.

FIG. 16. SEQUENCE 16 (SEQ ID NO: 16): blh. A codon harmonized version of blh, originating from the uncultured marine bacterium 66A03 (KR 1020160019480-A 32 19-Feb.-2016) and encoding for expression of 15,15′-dioxygenase.

FIG. 17. SEQUENCE 17 (SEQ ID NO:17): RDH12. A codon harmonized version of RDH12, originating from Homo sapiens and encoding for expression of retinol dehydrogenase.

FIG. 18. SEQUENCE 18 (SEQ ID NO:18): RDH12-short. A codon harmonized version of RDH12, originating from Homo sapiens and encoding for expression of a retinol dehydrogenase having the N-terminal transmembrane alpha-helix eliminated.

FIG. 19. SEQUENCE 19 (SEQ ID NO:19): yBBO. A codon harmonized version of YBBO, originating from E. coli and encoding for expression of an oxidoreductase.

FIG. 20. SEQUENCE 20 (SEQ ID NO:20): rdh12-His6. A codon harmonized version of RDH12, originating from Homo sapiens and encoding for expression of retinol dehydrogenase with a hexahistidine affinity tag (SEQ ID NO:39).

FIG. 21. SEQUENCE 21 (SEQ ID NO:21): yBBO. A codon harmonized version of YBBO, originating from E. coli and encoding for expression of an oxidoreductase hexahistidine affinity tag (SEQ ID NO:39).

FIG. 22. SEQUENCE 22 (SEQ ID NO:22): CRT1.2 (crtE→blh→crtY→rdh12). An operon containing two genes in the beta carotene pathway and the gene for conversion of beta carotene to retinol, with codon harmonized versions of the crtE, bib, crtY and RDH12 genes. These genes encode for the expression of GGPP synthase, 15,15′-dioxygenase, lycopene cyclase, and human retinol dehydrogenase. The sequence was modified to maintain the −1 spacing while eliminating the potential to add an uncleaved methionine to the beginning of the sequence and thereby altering protein production and/or activity.

FIG. 23. SEQUENCE 23 (SEQ ID NO:23): CRT1.2 (crtE→blh→crtY→rdh12). An operon containing two genes in the beta carotene pathway and the gene for conversion of beta carotene to retinol, with codon harmonized versions of the crtE, blh, crtY and RDH12-his6 genes. These genes encode for the expression of GGPP synthase, 15,15′-dioxygenase, lycopene cyclase, and his6-labeled (“his6” is disclosed as SEQ ID NO:39) human retinol dehydrogenase. The sequence was modified to maintain the −1 spacing while eliminating the potential to add an uncleaved methionine to the beginning of the sequence and thereby altering protein production and/or activity.

FIG. 24. SEQUENCE 24 (SEQ ID NO:24): CRT1.4 (crtE→blh→crtY→ybbo). An operon containing two genes in the beta carotene pathway and the gene for conversion of beta carotene to retinol, with codon harmonized versions of the crtE, blh, crtY and ybbo genes. These genes encode for the expression of GGPP synthase, 15,15′-dioxygenase, lycopene cyclase, and oxidoreductase. The sequence was modified to maintain the −1 spacing while eliminating the potential to add an uncleaved methionine to the beginning of the sequence and thereby altering protein production and/or activity.

FIG. 25. SEQUENCE 25 (SEQ ID NO:25): CRT1.4 (crtE→blh→crtY→ybbo-his6). An operon containing two genes in the beta carotene pathway and the gene for conversion of beta carotene to retinol, with codon harmonized versions of the crtE, blh, crtY and ybbo-his6 genes. These genes encode for the expression of GGPP synthase, 15,15′-dioxygenase, lycopene cyclase, and his6-labeled (“his6” is disclosed as SEQ ID NO:39) oxidoreductase. The sequence was modified to maintain the −1 spacing while eliminating the potential to add an uncleaved methionine to the beginning of the sequence and thereby altering protein production and/or activity.

FIG. 26. SEQUENCE 26 (SEQ ID NO:26): CRT2.2 (crtI→crtB→ispA) The CRT2.1 operon modified to maintain the −1 spacing while eliminating the potential to add an uncleaved methionine to the beginning of the sequence and thereby altering protein production and/or activity.

FIG. 27. SEQUENCE 27 (SEQ ID NO:27): crtE*. The crtE gene modified to maintain the −1 spacing while eliminating the potential to add an uncleaved methionine to the beginning of the sequence and thereby altering protein production and/or activity.

FIG. 28. SEQUENCE 28 (SEQ ID NO:28): crtY*. The crtY gene modified to maintain the −1 spacing while eliminating the potential to add an uncleaved methionine to the beginning of the sequence and thereby altering protein production and/or activity.

FIG. 29. SEQUENCE 29 (SEQ ID NO:29): crtI*. The crtI gene modified to maintain the −1 spacing while eliminating the potential to add an uncleaved methionine to the beginning of the sequence and thereby altering protein production and/or activity.

FIG. 30. SEQUENCE 30 (SEQ ID NO:30): RDH12-short. The amino acid sequence for a retinol dehydrogenase having the N-terminal transmembrane alpha-helix eliminated.

FIG. 31. A schematic of the mevalonate pathway is depicted.

FIG. 32. A schematic of the beta-carotene and retinol pathway is depicted.

FIG. 33. The figure depicts the design of a biofilm bioreactor designed for the synthesis and extraction of isoprenoids, carotenoids, and retinoids.

FIGS. 34A and B. The figure shows GC-MS data demonstrating retinol and retinoic acid production by M. atlanticus. Full spectrum of the gas chromatogram is shown in panel A and the selective reaction monitoring for retinol and retinoic acid is shown in panel B of retinol and retinoic acid standards and hexane extracts from the retinoid producing strains.

FIG. 35. The figure shows the UV-vis absorbance spectra of solvent overlays collected from retinoid-producing M. atlanticus cultures compared to standards of retinoic acid and retinal. Knocking out the native wax ester carbon storage pathway leads to increased retinol production.

FIG. 36. The figure shows a plot of 360 nm UV-Vis absorbance data for the solvent extract of retinol-producing M. atlanticus cultures. The increase in absorbance over time is indicative of retinoid production. Refreshing the medium in the biofilm retinol sample reinitiates retinoid production.

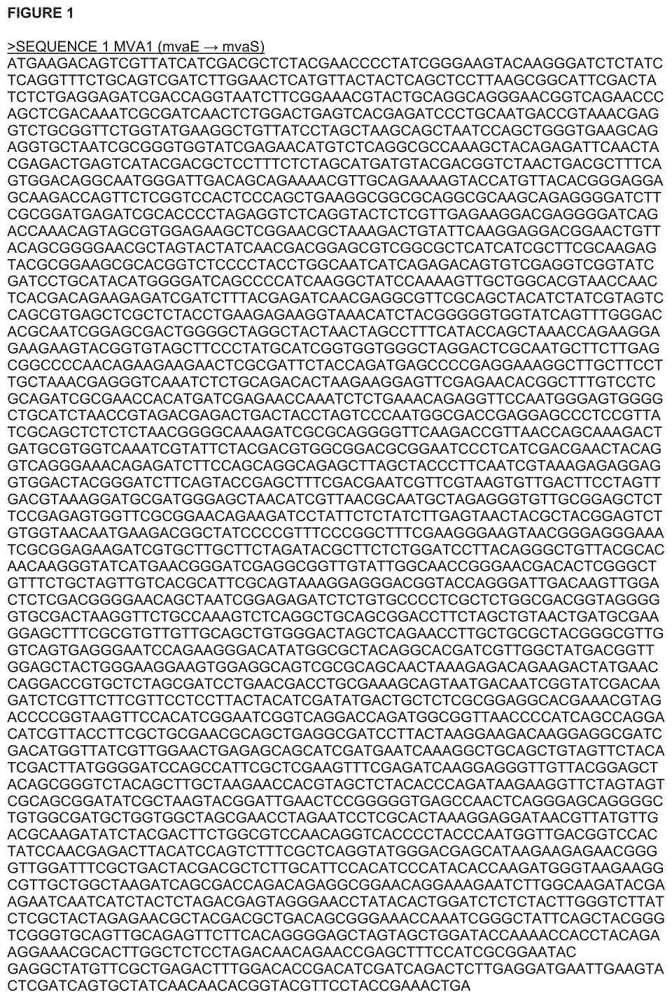

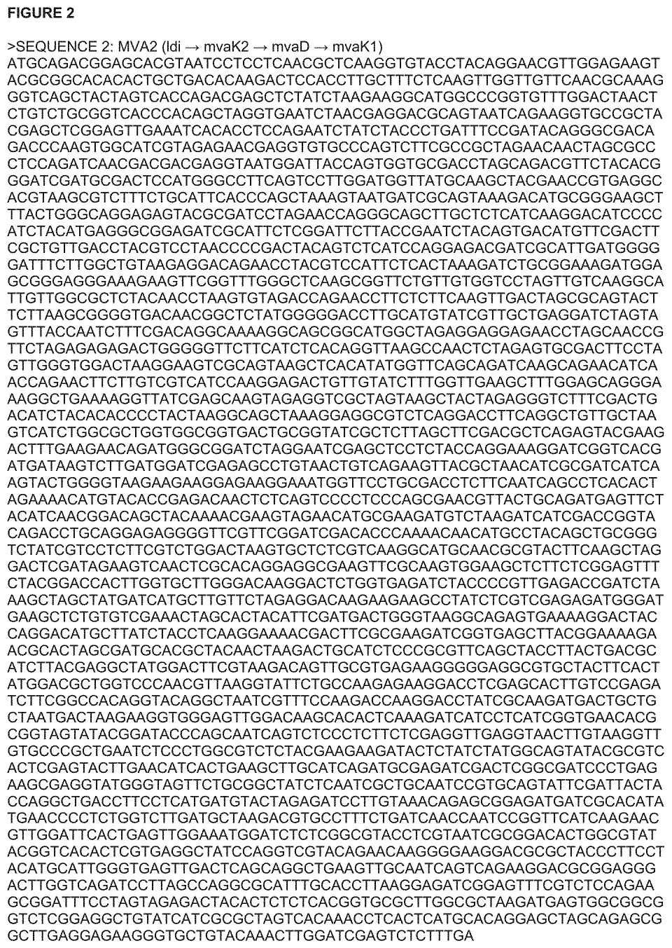

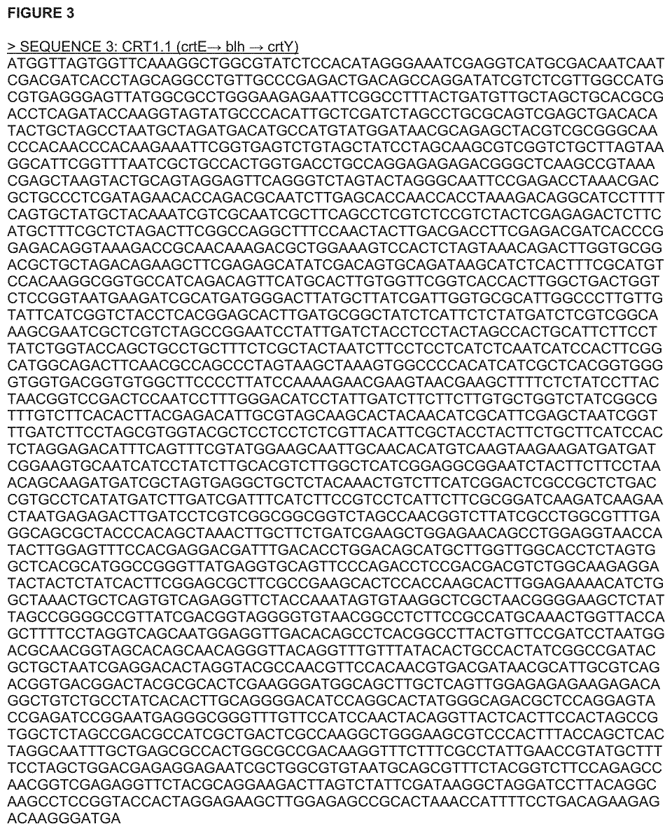

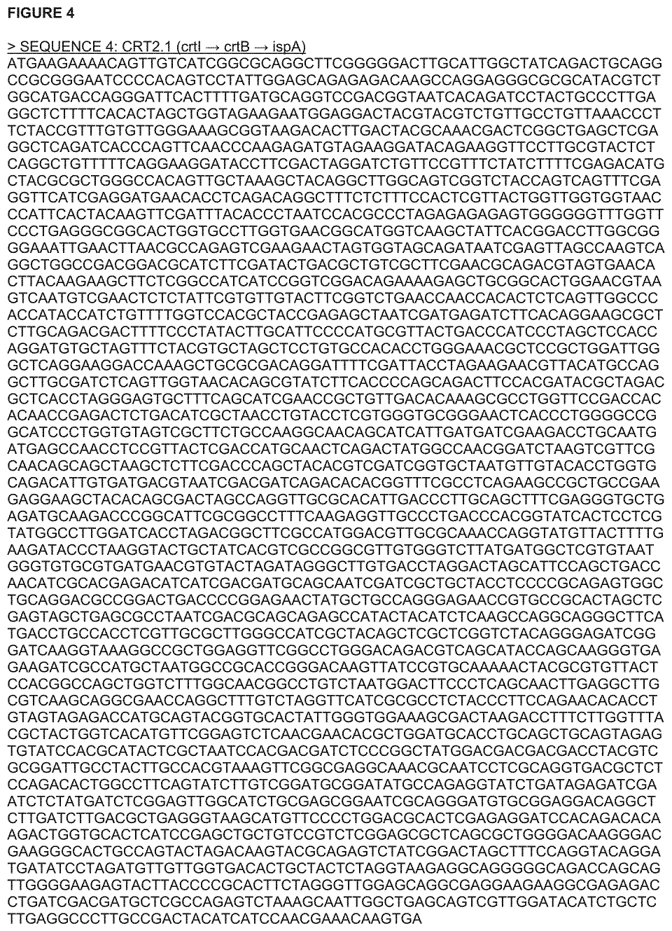

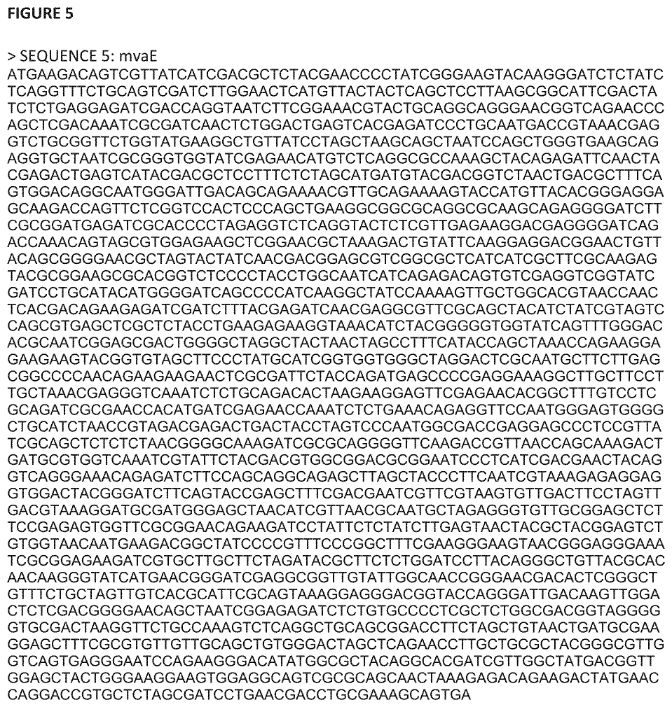

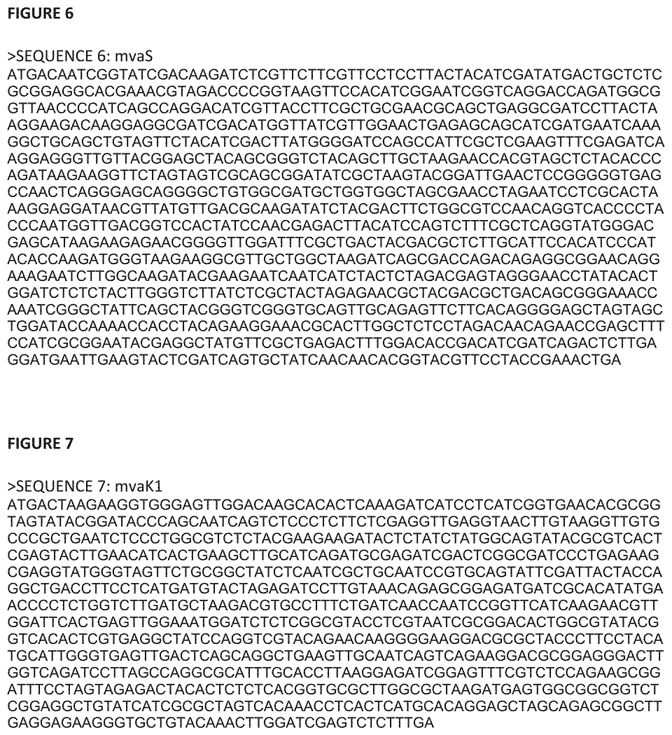

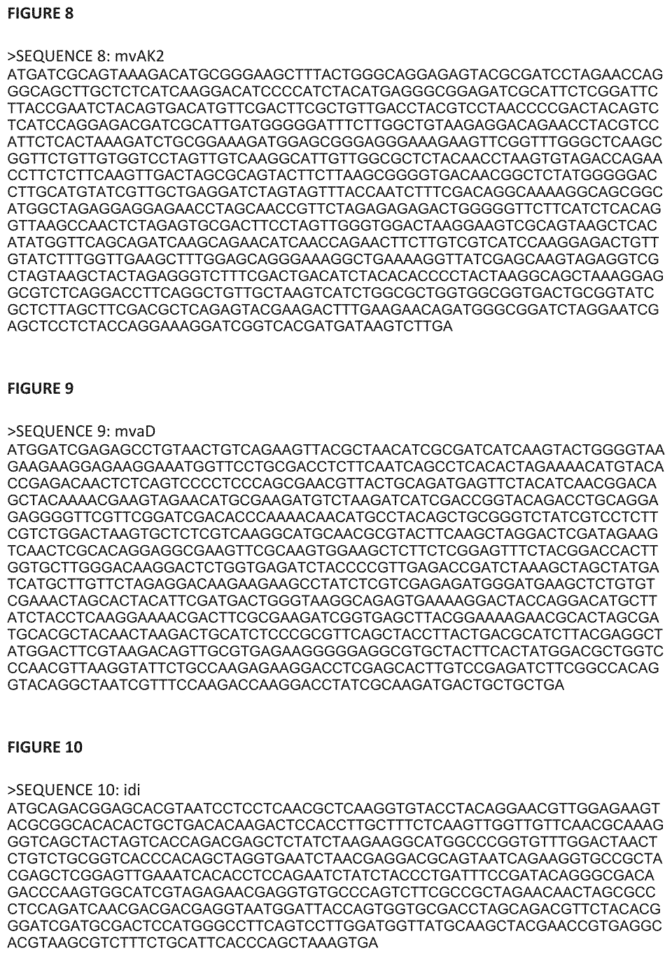

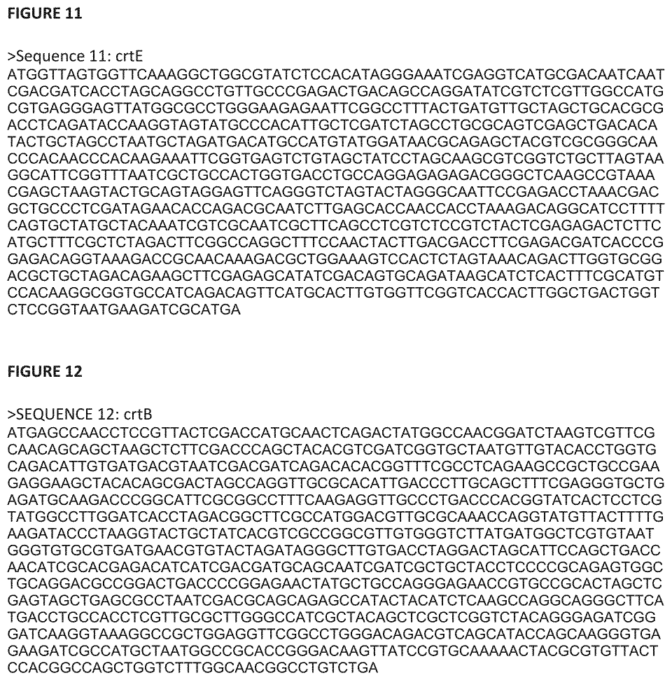

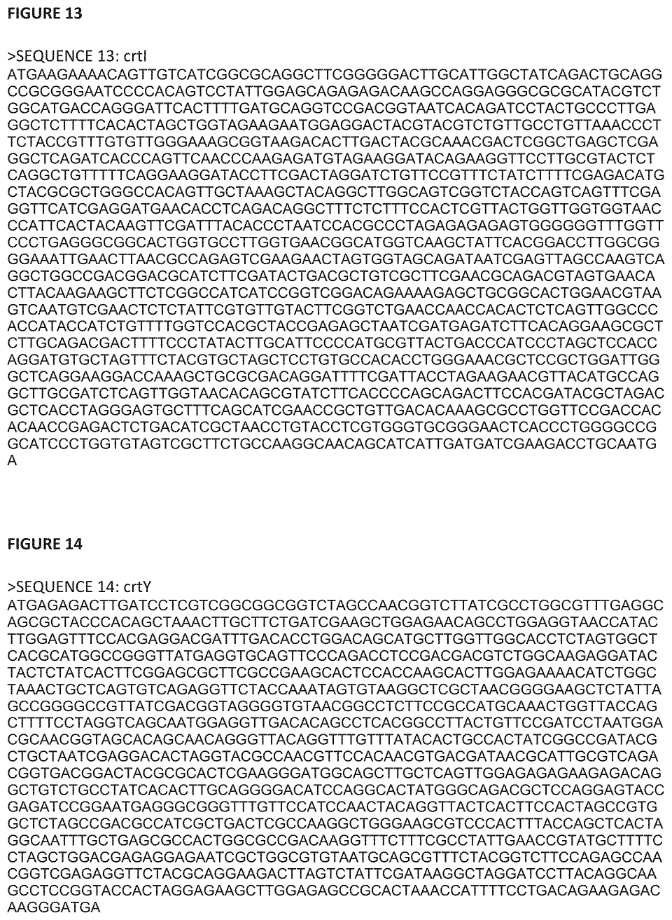

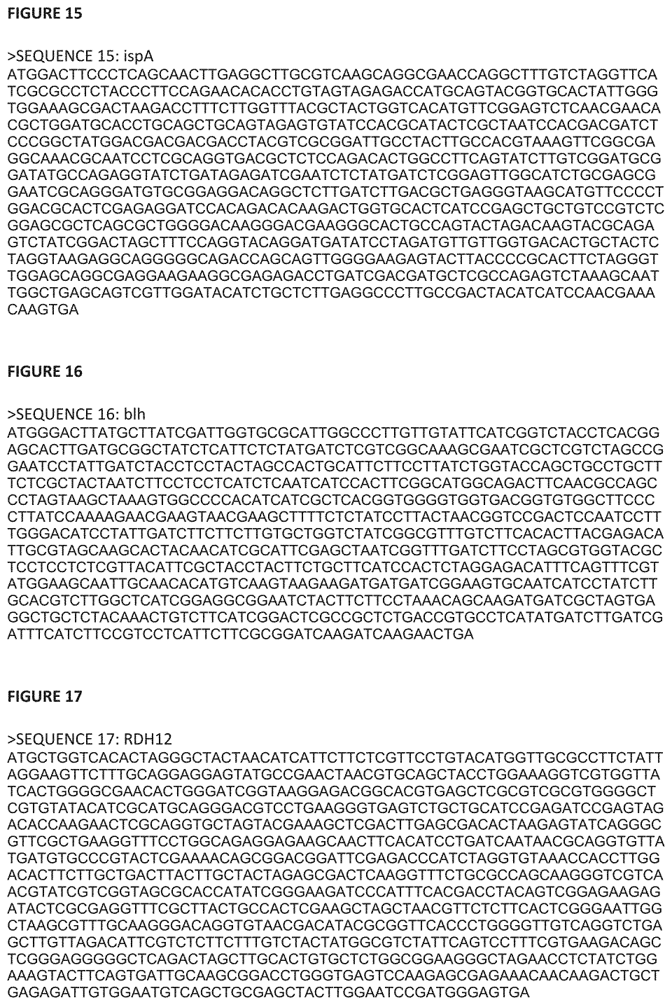

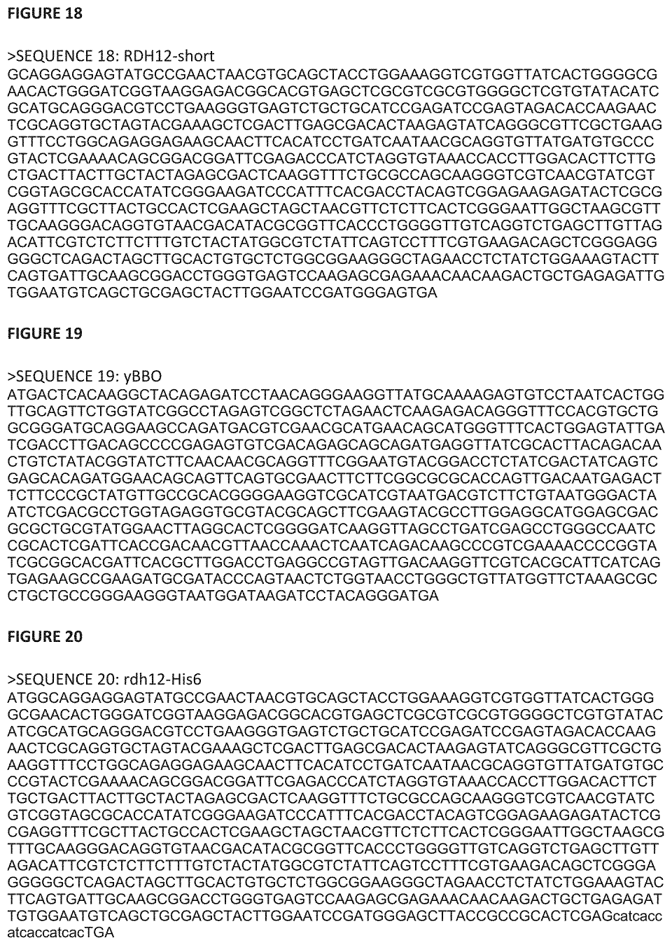

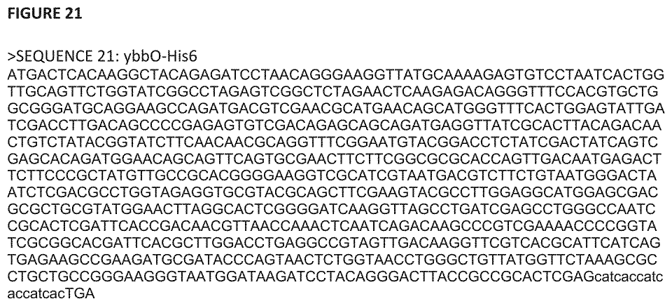

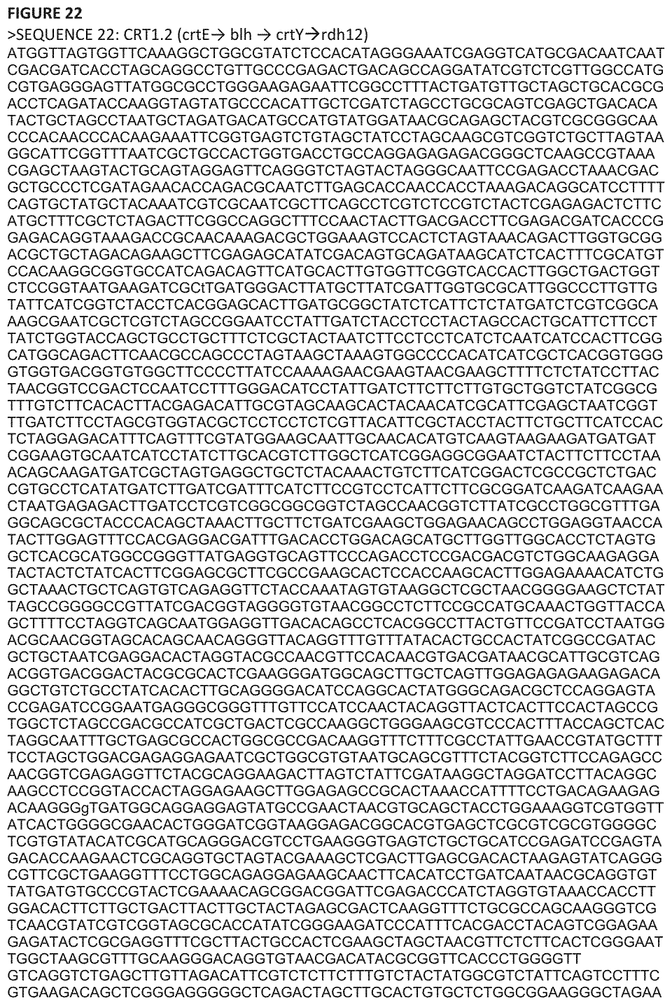

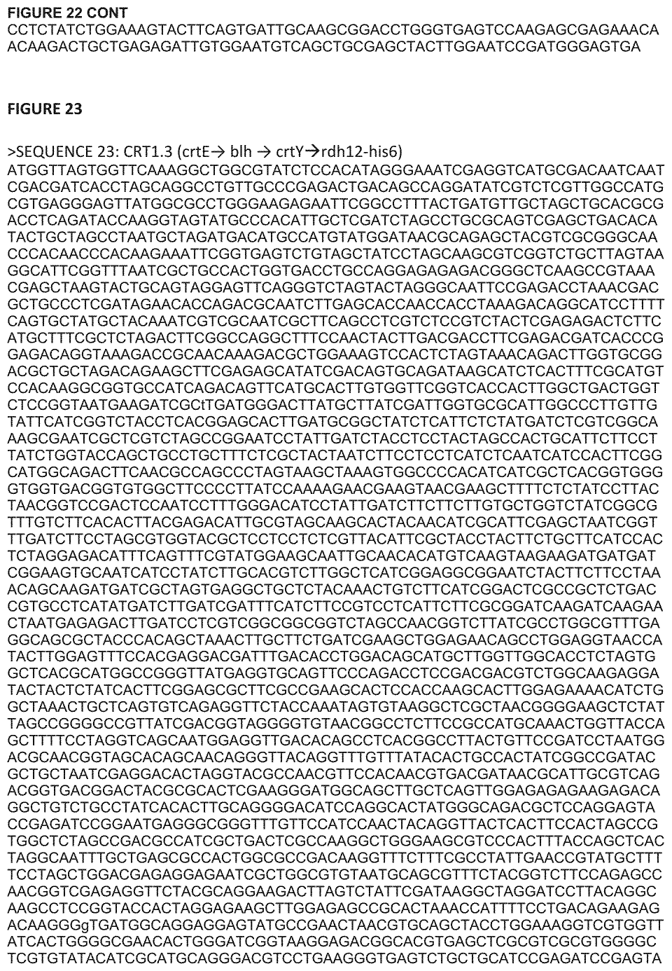

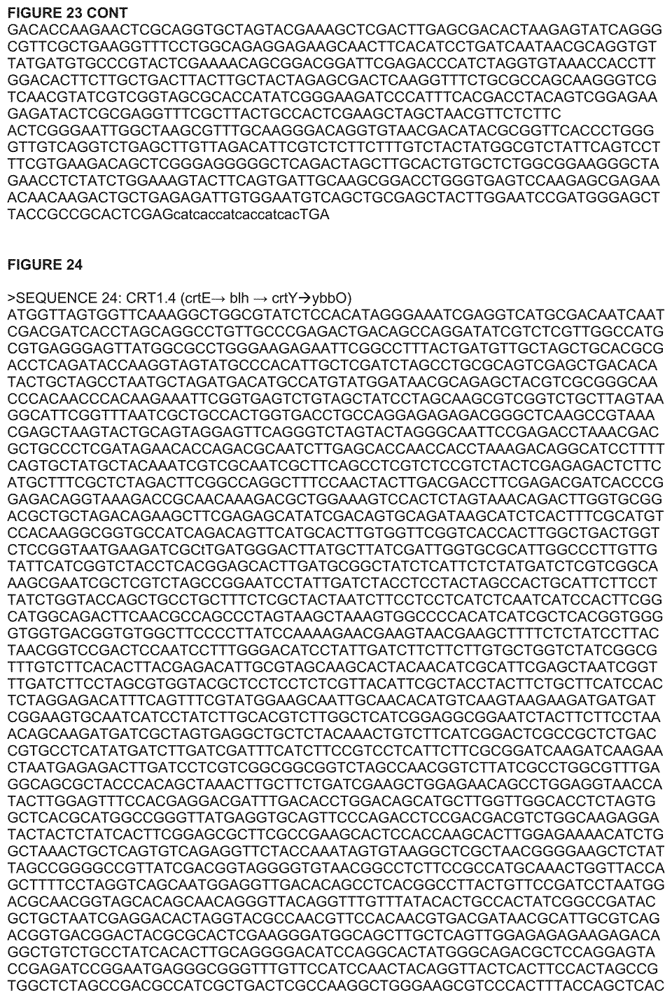

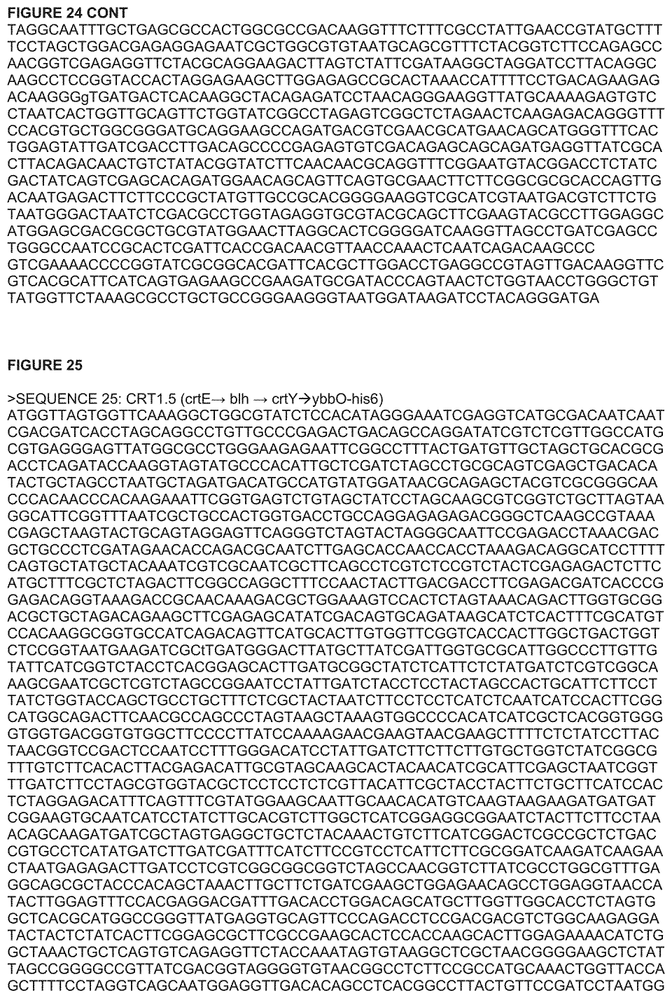

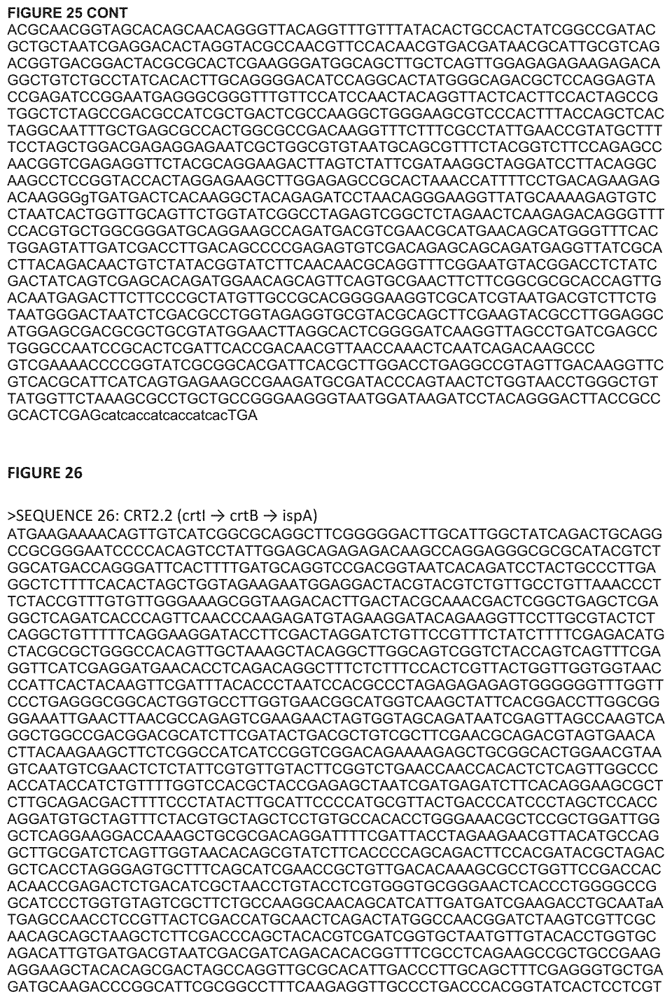

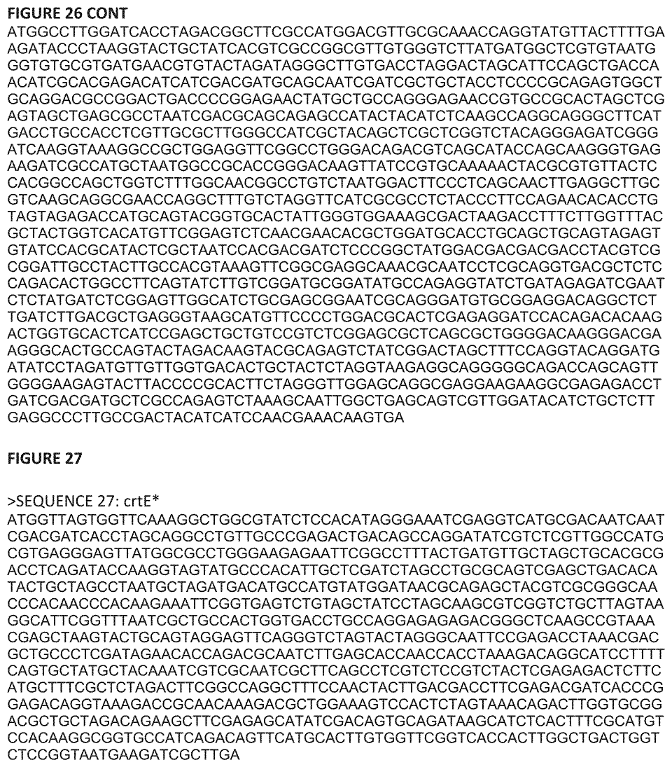

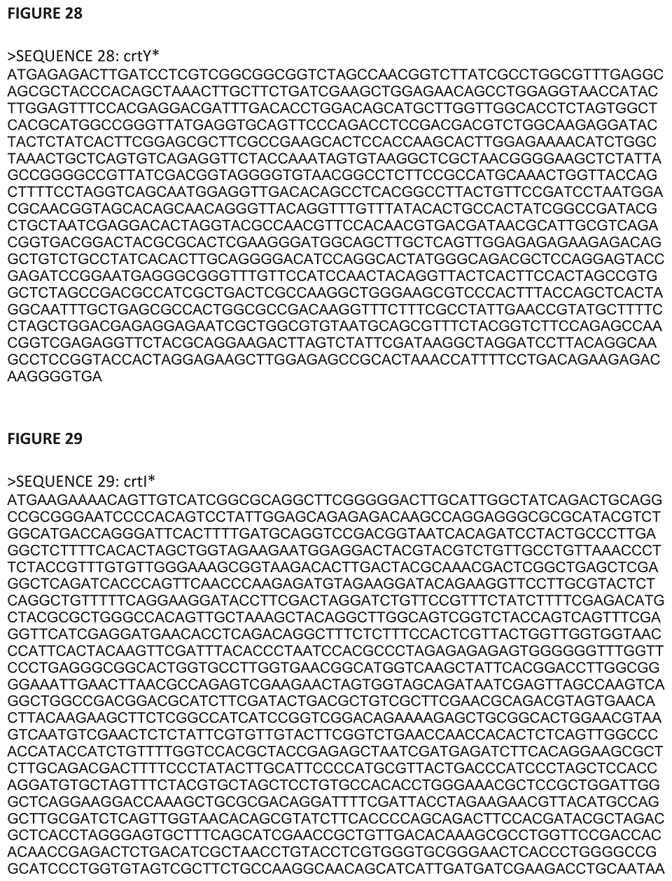

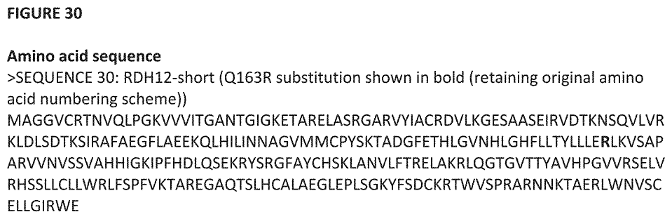

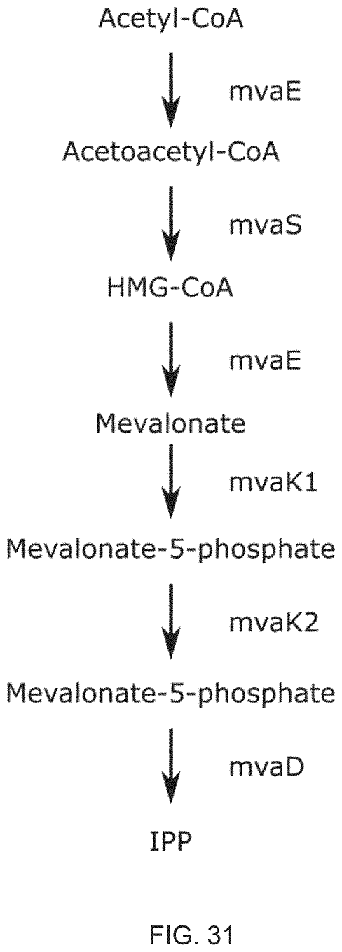

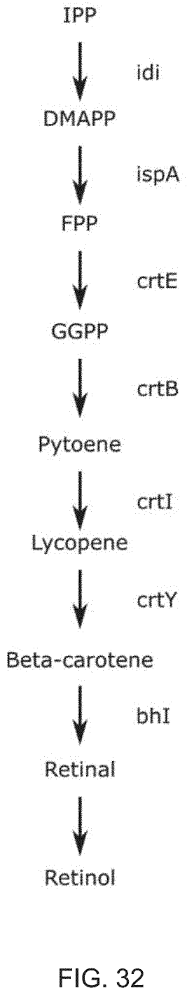

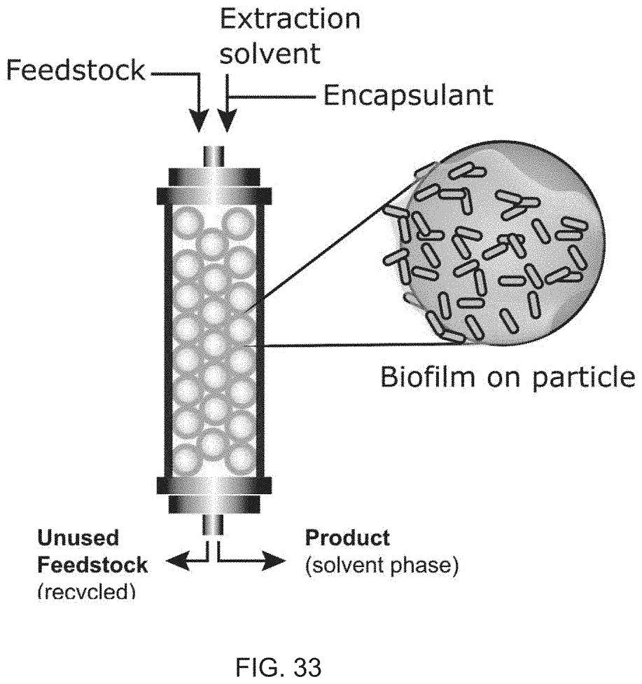

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise.

It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As described herein, the present invention encompasses genetically-engineered hydrocarbonoclastic microorganisms specifically engineered to synthesize isoprenoids, carotenoids, and retinoids in a biofilm or biofilm bioreactor in an efficient manner with high yield and purity. The majority of isoprenoids, carotenoids, and retinoids are not water-soluble, which presents challenges to traditional fermentative biosynthesis. Using a biofilm bioreactor to produce these molecules can enable more efficient, as well as better quality (e.g., less contamination or increased purity relative to other traditional production methods) product synthesis by using hydrophobic or non-polar solvents such as hexanes, decane, dodecane, oleic acid, or vegetable oils to extract the molecules. In this style of biofilm bioreactor, cells/microorganisms grow on the surface of small particles such as beads (˜10-500 microns—e.g., about 10, 20, 30 et seq. up to about 100, 200, 300, 400 or 500 microns) that are packed into a column. Suitable columns are known to those skilled in the art. Growth media containing a feedstock is circulated through the column and the cells in the biofilm (that is the biofilm comprising the genetically-engineered hydrocarbonoclastic cells as described herein) convert the feedstock into the product they have been engineered to produce (e.g., isoprenoids). The extraction solvent is introduced into the bioreactor and is allowed to contact the biofilm comprising the genetically-engineered microorganisms of the present invention, under conditions (e.g., flow rate and temperature) for a time suitable for maintaining contact with the genetically-engineered hydrocarbonoclastic organism present in the biofilm and removing/extracting/eluting the product to the hydrophobic phase. The eluted product is then captured for its specific use, or for further processing such as additional purification steps, concentration, mixture with other components/ingredients or processing for suitable storage.

Biofilms provide some inherent protection to cells against the toxic effects of both the product and the extraction solvent. Hydrocarbonoclastic organisms, microorganisms that degrade hydrocarbons, are particularly well suited for product synthesis in this type of bioreactor because in nature they often form biofilms directly on the surface of oil droplets in water. As a result, these organisms have developed a number of biological features including the active export of solvent molecules (Iksen 1998; Ramos 2002) and the production of biosurfactants (Raddadi 2017), which improve tolerance to non-polar solvents.

Many hydrocarbonoclastic organisms contain natural carbon storage pathways (Klauscher 2007; Manila-Perez 2010) that route excess carbon feedstocks to the production of wax esters for later use. To produce alternative products, the organism can be engineered to reroute this carbon storage pathway. Unlike model organisms such as E. coli, many hydrocarbonoclastic organisms do not have well developed procedures for introducing new metabolic pathways, which is a prerequisite for engineering the organism to produce a new pathway. Genetic systems have been developed to enable the engineering of several species of Marinobacter (Sonnenschein 2011: Bird 2018).

Unlike E. coli, many Marinobacter spp. and similar organisms contain pathways for metabolism of short chain fatty acids such as acetate. Consequently, a pathway for conversion of glucose to acetate does not need to be introduced into the target organism.

Described herein are methods for producing isoprenoids, including carotenoids and retinoids using Marinobacter species in a biofilm or biofilm bioreactor. More specifically, described herein is the complete pathway for the bioreactor synthesis of retinol. The retinol pathway is inclusive of the mevalonate and beta-carotene pathway, and the production of alternative products can be accomplished by using only a portion of this pathway. The pathway begins with a pool of acetyl-CoA. In Marinobacter and similar organisms, this pool of acetyl-CoA is part of the carbon storage pathway for the production of wax esters. In some embodiments of the invention, the host organism will be engineered to knock out the natural wax ester pathway.

The upper mevalonate pathway converts acetyl-CoA to mevalonate, as described in Jang et al., 2012. First the mvaE gene (Sequence 5), expressing acetyl-CoA acetyltransferase converts two acetyl-CoA to acetoacetyl-CoA, next the mvaS gene expresses HMG-CoA synthase, which produces β-Hydroxy β-methylglutaryl-CoA (HMG-CoA) from acetoacetyl-CoA and acetyl-CoA. In some embodiments, the alanine in position 110 of mvaS is substituted for a glycine, which can improve overall yield. The mvaE gene also yields a HMG-CoA reductase that converts HMG-CoA to mevalonate as the final step in the upper mevalonate pathway. The mvaE and mvaS genes (Sequence 6) originate from Enterococcus faecalis.

Next, the lower mevalonate pathway converts IPP from mevalonate, as described in Yoon et al., 2009. In the lower mevalonate pathway, the mvaK1 gene (Sequence 7) encodes mevalonate kinase, which produces mevalonate-5-phosphate from mevalonate and ATP. Next the mvaK2 gene (Sequence 8) encodes phosphomevalonate kinase, which converts mevalonate-5-phosphate to mevalonate-5-pyrophosphate. The mvaD gene (Sequence 9) produces mevalonate-5-pyrophosphate decarboxylase that converts mevalonate-5-pyrophosphate to IPP. The mvaK1, mvaK2, and mvaD genes originate from Streptococcus pneumoniae ATCC 6314.

The beta-carotene pathway produces beta-carotene from IPP (Yoon 2007, Kang, 2005). In the first step, the idi gene (Sequence 10) encodes isopentenyl-PP isomerase which converts IPP to DMAPP (Yoon 2009). Next, the ispA gene (Sequence 15), encoding farnesyl diphosphate synthase, produces FPP from DMAPP and IPP. The crtE gene (Sequence 11) product (GGPP synthase) produces GGPP from FPP and IPP. Next, the crtB gene (Sequence 12) product (phytoene synthase) produces phytoene from GGPP and the crtI gene (Sequence 13) product (phytoene desaturase) converts phytoene to lycopene. Finally, the crtY gene (Sequence 14) product (lycopene cyclase) converts lycopene to beta-carotene. The ipi and ispA genes originate from Escherichia coli str. K-12 substr. W3110. crtE, crtB, crtI, and crtY originate from Pantoea agglomerans KCCM 40420.

The conversion of beta-carotene into retinal is carried out by 15,15′-dioxygenase encoded by the blh gene. The conversion of retinal to retinol occurs spontaneously. Blh (Sequence 16) originates from the uncultured marine bacterium 66A03 (KR 1020160019480-A 32 19 Feb. 2016).

A reductase enzyme can be used to actively reduce retinal to retinol. A suitable enzyme is the oxidoreductase encoded by the ybbO gene (Sequence 19) originating from E. coli (Jang, 2015) In an alternative embodiment, human retinol dehydrogenase, encoded by the gene RDH12 (Sequence 17) is used to convert retinal to retinol. Human retinol dehydrogenase 12 is an integral membrane protein. While RDH12 has been shown to improve the selective production of retinol vs retinal in Saccharomyces cervisiae (Lee, 2022), attempts at bacterial expression of RDH12 have failed to yield active enzyme (Burgess-Brown, 2008) due to poor solubility. It was identified that the RDH12 enzyme has an N-terminal transmembrane alpha-helix, which likely contributes to the poor solubility of the enzyme. A gene to express a modified version of the RDH12 protein eliminating the first 26 amino acids from the N-terminus was designed. (Sequence 30). While the DNA database of Japan listing for RDH12, accension number BC025724, has a glutamine (Q) at amino acid 163, many other retinol dehydrogenases as well as the Uniprot listing, Q96N48, for RDH12 have an arginine at amino acid 163. Different embodiments of the invention can include either of these sequences.

To facilitate the optimal expression of each gene, the nucleic acid sequences were optimized for the host organism through codon harmonization. Codon harmonization evaluates codon usage in the donor organism and the host organism and optimizes the codon distribution in the introduced gene for the host. Codon harmonization was carried out using the CodonWizard software (Rehbein 2019) for each gene. Codon usage for coding nucleic acid sequences for both the host and donor organisms were determined and then the codon harmonization algorithm was applied to the donor nucleic acid sequence.

For ease of synthesis and to optimize protein expression, operons were designed with 4 groupings of genes, having a −1 spacing between genes within the operon. These groupings are: MVA1 (Sequence 1): mvaE→mvaS; MVA2 (Sequence 2): idi→mvaK2→mvaD→mvaK1; CRT1(Sequence 3): crtE→bhl→crtY; CRT2 (Sequence 4): crtI→crtB→ispA.

To include RDH12 or ybbO, the gene was added to the CRT1 operon, CRT1.2 crtE→bhI→crtY→RDH12 or CRT1.3 (Sequence 3): crtE→bhI→crtY→ybbO. In some embodiments individual gene sequences are modified to include a HIS-6 tag (SEQ ID NO:39) on the protein to allow for more facile characterization of protein expression.

Synthetic operons CRT1.2-CRT1.4 and CRT2.2 were created as above with the crtE*, crtY*, and crtI* genes were modified to maintain the −1 spacing while eliminating the potential to add an uncleaved methionine to the beginning of the sequence and thereby altering protein production and/or activity.

It may be desirable to reorder the genes within or among these synthetic operons or to place each gene under the control of a separate genetic control elements (e.g., promoter, RBS, transcriptional terminator) to achieve optimal expression levels for each of these genes and production of the corresponding gene products.

These genes are assembled using Gibson assembly or a comparable technique known to those skilled into the art and cloned into a single plasmid in E. coli. In some embodiments, each grouping will be under the control of a constitutive promoter while in other embodiments each gene will have an individual constitutive promoter. The lac promoter is preferred, with tac, trp and osmY as additional possible promoters. Other promoters that are suitable for use in stationary phase and compatible with the specific hydrocarbonoclastic organisms could also be used. This plasmid is introduced to the host organism (e.g., M. atlanticus) through conjugation with E. coli, as described in Bird, 2018. To enable longer term product synthesis in a biofilm, the use of an integrative plasmid allows for integration of the genes into the host genome through homologous recombination.

The method for synthesizing retinol in a bioreactor begins with the preparation of the bioreactor. An overnight culture of the hydrocarbonoclastic organism containing the engineered isoprenoid pathway is incubated in a biofilm-promoting media with a particulate biofilm support material (e.g., glass, plastic, carbon, wood) for 6-24 hours to seed these particles with biofilm. The biofilm on the support particles is lyophilized and then mixed with sterile support material and introduced into the bioreactor at about a 1:10 ratio in a packed bed configuration. The bioreactor is initially run for about 24 to 100 hours to promote the formation of a stable, productive biofilm. After that initial period, media containing a carbon source and other nutrients is continuously circulated through the reactor with the carbon source converted to product by the biofilm. Periodically, a hydrophobic extraction solvent is introduced into the reactor to remove the product from the cells in the biofilm. Suitable solvents include hexane, decane, dodecane, squalane, farnesene, liquid fatty acids such as oleic acid, mineral oil and vegetable oils. In some embodiments the organic solvent will be introduced as a plug that fills the entire cross-section of the bioreactor, while in other embodiments the organic solvent will be dispersed in small droplets and introduced into the bioreactor along with the medium.

The solvent may, in some embodiments, contain additional surfactants or encapsulants that protect oxidation-sensitive products such as retinal or retinol. These encapsulants may include molecules such as cyclodextrin (Semenova 2002) or may include lipid molecules such as phosphatidylcholine or cholesterol that can be used to encapsulate the product molecule in a liposome (Singh 1998). This immediate encapsulation has the benefit of improving product activity by inhibiting oxidation that may take place through subsequent purification steps. The hydrophobic solvent phase can be separated from the water phase using a passive phase separator consisting of a hydrophobic membrane. In some embodiments the product is then purified from the solvent phase through distillation or lyophilization. In other embodiments, filtration is used to separate the product based on size. In yet further embodiments, the extraction solution can be incorporated into the final/end product.

### Example 1: Engineering of M. atlanticus to Produce Retinoids

To demonstrate retinol production in M. atlanticus, the pBBR-mev and pSEVA.652.ret plasmids were introduced into WT M. atlanticus or a M. atlanticus strain where the wax ester carbon storage pathway was knocked out, ΔΔM. atlanticus (Bird, et al 2018): pBBR-mev contains the pBBR1-MCS2 plasmid backbone with the mevalonate pathway inserted at the multiple cloning site. pSEVA.651.ret contains the pSEVA.651 plasmid backbone (Silva-Rocha, 2013) with the genes require to transform dimethylallylpyrophosphate, the terminal product of the mevalonate pathway, into retinol inserted at the multiple cloning site.

In the pBBR1-MCS2 backbone, mvaE and mvaS were placed in a synthetic operon with a −1 spacing between the two genes where the last nucleotide of the mvaE stop codon was also the first nucleotide of the ATG start codon for mvaS. This synthetic operon was placed under the control of a constitutive lac promoter (pLacQI) with the sequence: TGGTGCAAAACCTTTCGCGGTATGGCATGATAGCGCC (SEQ ID NO:31) and ribosome binding site (B0064) with the sequence: tactagagaaagaggggaaatactag (SEQ ID NO:32). A transcriptional terminator (L3S3P21) was placed after mvaS with the sequence:

(Chen 2013)
(SEQ ID NO: 33)
CCAATTATTGAAGGCCTCCCTAACGGGGGGCCTTTTTGTTTCTGGTCTC
CC.

A second synthetic operon contained, in the following order: idi, mvaK2, mvaD, and mvaK1. Each gene had a −1 spacing between each where the last nucleotide of the stop codon was the first nucleotide of the following start codon. This second operon was placed under the control of a constitutive lac promoter with the sequence: TTTACACTTTATGCTTCCGGCTCGTATGTTG (SEQ ID NO:34) with a ribosome binding site (B0030) with the sequence: TAGTACATTAAAGAGGAGAAATAGTAC (SEQ ID NO:35).

Within the pSEVA.651 backbone, crtE, blh, crtY, and the truncated RDH12 were arranged into a synthetic operon (CRT1.2) with a −1 spacing between each gene where the last nucleotide of the stop codon was the first nucleotide of the following start codon. This synthetic operon was placed under the control of a constitutive lac promoter and RBS with the following sequence: TTTACACTTTATGCTTCCGGCTCGTATGTTGTGTGGAATTGTGAGCGTCTAGTAGA AGGAGGAGATCTGGATCCAT (SEQ ID NO:36). A transcriptional terminator (L3S2P21) was placed after RDH12 with the sequence:

(Chen, 2013)
(SEQ ID NO: 37)
CTCGGTACCAAATTCCAGAAAAGAGGCCTCCCGAAAGGGGGGCCTTTTTT
CGTTTTGGTCC.

In a second operon (CRT2.1), crtI, crtB, and ispA were arranged in the listed order with a −1 spacing between each gene as described above. This operon was placed under the control of a constitutive lac promoter and ribosome binding with the sequence: aaaacctttcgcggtatggcatgatagcgcccggaagagagtcaattcagggaggtgaat (SEQ ID NO:38).

The pBBR-mev plasmid was introduced into WT M. atlanticus or ΔΔ M. atlanticus via conjugation using the diaminopimelic acid (DAP) auxotroph donor strain E. coli WM3064 that was transformed with pBBR-mev. ΔΔ M. atlanticus colonies containing the pBBR-mev plasmid were selected for in the presence of kanamycin and absence of DAP. Successful introduction of the pBBR-mev plasmid into M. atlanticus was confirmed via PCR and subsequent DNA sequencing. The pSEVA.651.ret plasmid was then transformed into E. coli WM3064, and the selection process repeated with the addition of gentamicin in the selection agar. Successful introduction and maintenance of both pBBR-mev and pSEVA.651.ret plasmids were confirmed by PCR and DNA sequencing.

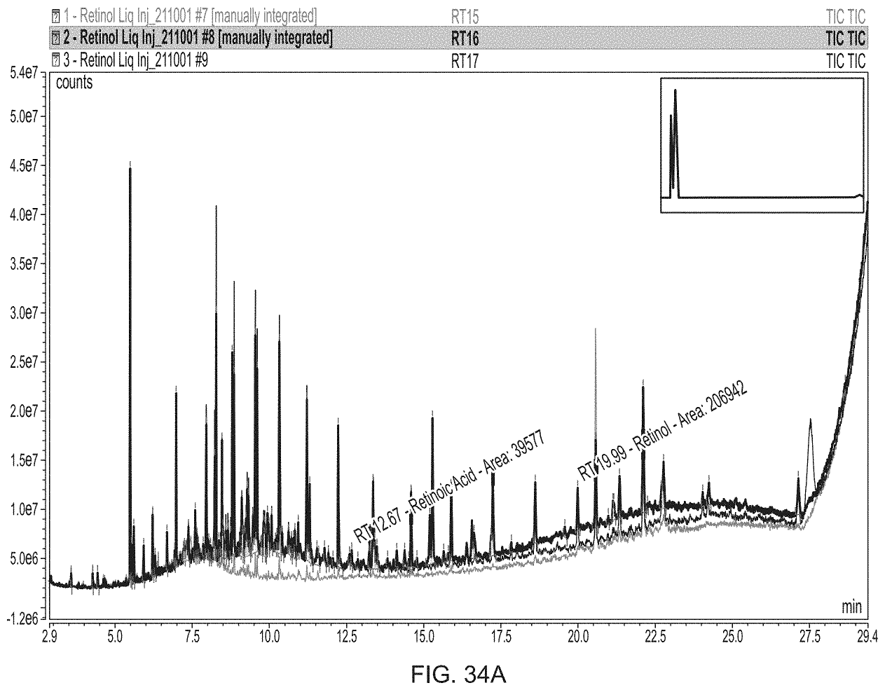

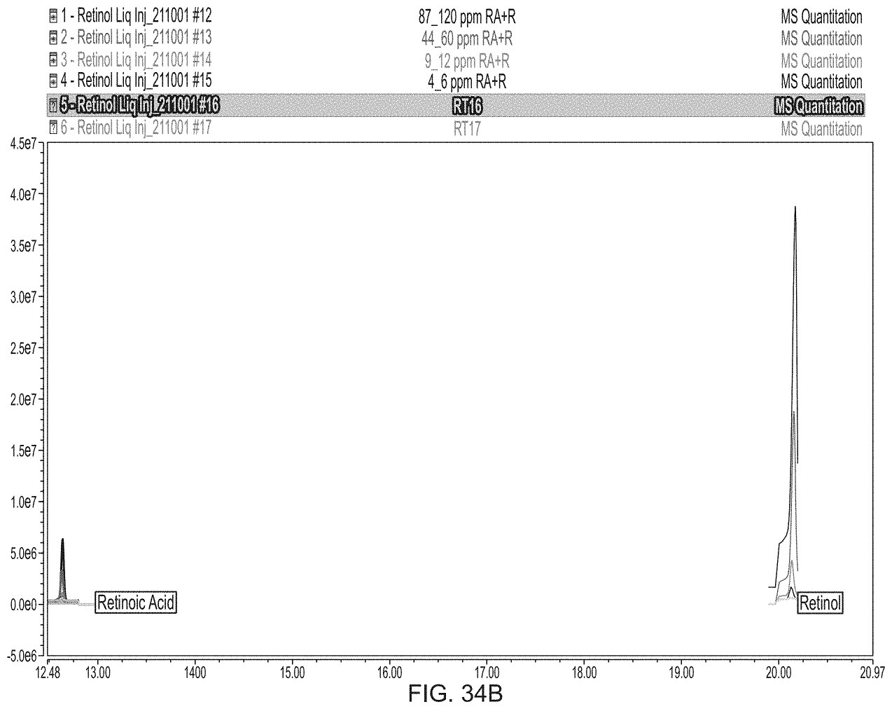

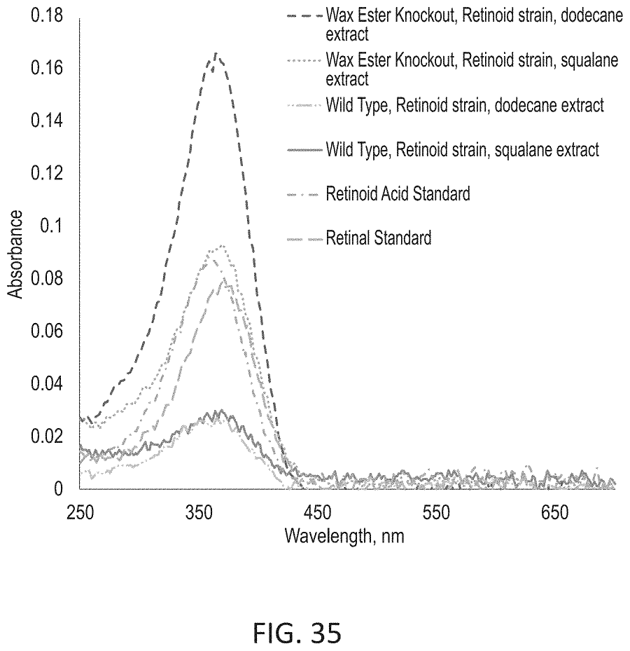

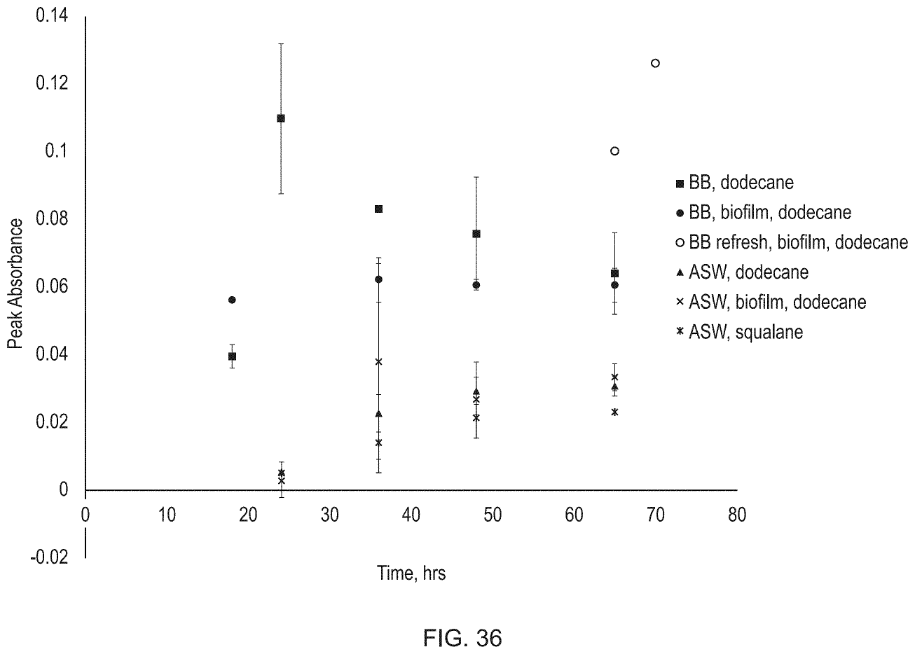

To evaluate retinoid production, overnight cultures of individual colonies were grown. The overnight culture was diluted 1:100 in a saltwater medium and grown for 12 hrs. The cells from the overnight culture were pelleted, lyophilized, and then treated with n-hexane to extract retinoids. Successful production of retinoic acid and retinol was observed by GC-MS, FIG. 34 and UV-Vis, FIG. 35.

### Example 2: Extraction of Retinol in Squalane, Dodecane

To demonstrate the continuous production of retinoids and their extraction into hydrophobic organic solvents, cultures of a M. atlanticus strain engineered to contain plasmids for both the mevalonate and retinol pathways are grown overnight in a rich medium (e.g., a rich saltwater medium) from a single colony. Both a native wax ester-producing strain and a strain where two wax esters producing genes have been knocked out were evaluated. These cultures are diluted 1:100 in 3 mL fresh media in 16 mm pyrex culture tubes. Growth conditions with a minimal artificial sea water media and a rich media were tested as well as both in biofilm forming conditions with glass beads and conditions for planktonic growth. In some samples, an extraction solvent of between 0.5-1 mL of organic solvent was used as an overlay to continuously remove retinol from the cultures. Samples were grown for at least 12 h and up to 48 h. Samples of the extraction solution were taken over time. Retinoids production was characterized by UV-Vis. FIG. 34 shows retinoid extraction into both dodecane and squalane. The kinetics of retinol production are shown in FIG. 35. While retinol concentration peaked at about 12 h, retinol production was able to be reinitiated in the biofilm samples upon addition of additional nutrients as described herein.

### Example 3: Production of Retinol in a Biofilm Bioreactor

Cultures of a M. atlanticus strain with plasmids for both the mevalonate and retinol pathways are grown over night in rich, saltwater media. A culture flask containing a silica solid support and saltwater medium designed to promote biofilm formation is inoculated with the overnight culture. The following day, the biofilm-coated beads are transferred into 10 mL biofilm bioreactors. Saltwater media with succinate as a carbon source was circulated through the bioreactor under closed-loop flow control, maintaining a constant flow rate of between 1-6 mL/min. Periodically (between every 3-6 h) hexane was flushed through the reactor to extract retinoids. Hexane extracts were concentrated by lyophilization and characterized for retinoids by absorbance.

While this invention has been particularly shown and described with references to preferred embodiments thereof, it will be understood by those skilled in the art that various changes in form and details may be made therein without departing from the scope of the invention encompassed by the appended claims.

The following references are herein incorporated by reference in their entirety.

### REFERENCES

Bird, L. J., Wang, Z., Malanoski, A. P., Onderko, E. L., Johnson, B. J., Moore, M. H., Phillips, D. A., Chu, B. J., Doyle, J. F., Eddie, B. J. and Glaven, S. M., 2018. Development of a genetic system for Marinobacter atlanticus CP1 (sp. nov.), a wax ester producing strain isolated from an autotrophic biocathode. Frontiers in microbiology, 9, p.3176.
Burgess-Brown, N. A., Sharma, S., Sobott, F., Loenarz, C., Oppermann, U. and Gileadi, O., 2008. Codon optimization can improve expression of human genes in Escherichia coli: A multi-gene study. Protein expression and purification, 59(l), pp. 94-102.
Chen, Y. J., Liu, P., Nielsen, A. A., Brophy, J. A., Clancy, K., Peterson, T. and Voigt, C. A., 2013. Characterization of 582 natural and synthetic terminators and quantification of their design constraints. Nature method, 10(7), pp. 659-664.
Daugulis A J. 1997. Partitioning bioreactors. Curr Opin Biotechnol 8(2). 169-174.
Gauthier, M. J., Lafay, B., Christen, R., Fernandez, L., Acquaviva, M., Bonin, P. and Bertrand, J. C., 1992. Marinobacter hydrocarbonoclasticus gen. nov., sp. nov., a new, extremely halotolerant, hydrocarbon-degrading marine bacterium. International Journal of Systematic and Evolutionary Microbiology, 42(4), pp.568-576.
Handley, K. M. and Lloyd, J. R., 2013. Biogeochemical implications of the ubiquitous colonization of marine habitats and redox gradients by Marinobacter species. Frontiers in microbiology, 4, p.136.
Isken, S. and de Bont, J. A., 1998. Bacteria tolerant to organic solvents. Extremophiles, 2(3), pp.229-238.
Jang, J., Xian, M., Su, S., Zhao, G., Nie, Q., Jiang, X., Zheng, Y., Liu, Wei. Enhancing Production of Bio-Isoprene Using Hybrid MVA Pathway and Isoprene Synthase in E. coli. PLoS one, 2012, 7, 4, e33509.
Jang, H. J., Ha, B. K., Zhou, J., Ahn, J., Yoon, S. H. and Kim, S. W., 2015. Selective retinol production by modulating the composition of retinoids from metabolically engineered E. coli. Biotechnology and Bioengineering, 112(8), pp.1604-1612.
Kalscheuer, R., Stöveken, T., Malkus, U., Reichelt, R., Golyshin, P. N., Sabirova, J. S., Ferrer, M., Timmis, K. N. and Steinbüchel, A., 2007. Analysis of storage lipid accumulation in Alcanivorax borkumensis: evidence for alternative triacylglycerol biosynthesis routes in bacteria. Journal of bacteriology, 189(3), pp.918-928.
Kang, M.; Yoon, S.; Lee, Y., Lee, S.; Kim, J.; Jung, K.; Shin, Y.; Kim, S. Enhancement of Lycopene Production in Escherichia coli by Optimization of the Lycopene Synthetic Pathway. (2005) J. Microbio. Biotechnol. 15, 4, 880-886.
Lee, Y. G., Kim, C., Sun, L., Lee, T. H. and Jin, Y. S., 2022. Selective production of retinol by engineered Saccharomyces cerevisiae through the expression of retinol dehydrogenase. Biotechnology and Bioengineering, 119(2), pp. 399-410.
Malinowski J J. 2001 Two-phase partitioning bioreactors in fermentation technology. Biotechnol Adv 19: 525-538.
Manilla-Perez, E., Lange, A. B., Hetzler, S. and Steinbüchel, A., 2010. Occurrence, production, and export of lipophilic compounds by hydrocarbonoclastic marine bacteria and their potential use to produce bulk chemicals from hydrocarbons. Applied microbiology and biotechnology, 86(6), pp.1693-1706.
Raddadi, N., Giacomucci, L., Totaro, G. and Fava, F., 2017. Marinobacter sp. from marine sediments produce highly stable surface-active agents for combatting marine oil spills. Microbial cell factories, 16(1), pp.1-13.
Rehbein, P., Berz, J., Kreisel, P. and Schwalbe, H., 2019. “CodonWizard”—An intuitive software tool with graphical user interface for customizable codon optimization in protein expression efforts. Protein expression and purification, 160, pp.84-93.
Semenova, E. M., Cooper, A., Wilson, C. G. and Converse, C. A., 2002. Stabilization of all-trans-retinol by cyclodextrins: a comparative study using HPLC and fluorescence spectroscopy. Journal of inclusion phenomena and macrocyclic chemistry, 44(1), pp.155-158.
Silva-Rocha, R., Martínez-García, E., Calles, B., Chavarría, M., Arce-Rodríguez, A., de Las Heras, A., Paez-Espino, A. D., Durante-Rodríguez, G., Kim, J., Nikel, P. I. and Platero, R., 2013. The Standard European Vector Architecture (SEVA): a coherent platform for the analysis and deployment of complex prokaryotic phenotypes. Nucleic acids research, 41(D1), pp.D666-D675.
Singh, A. K. and Das, J., 1998. Liposome encapsulated vitamin A compounds exhibit greater stability and diminished toxicity. Biophysical chemistry, 73(1-2), pp.155-162.
Sonnenschein, E. C., Gardes, A., Seebah, S., Torres-Monroy, 1., Grossart, H. P. and Ullrich, M. S., 2011. Development of a genetic system for Marinobacter adhaerens HP15 involved in marine aggregate formation by interacting with diatom cells. Journal of microbiological methods, 87(2), pp.176-183.
Yoon, S H, Park H M, Kim J E, Lee S H, Choi M S, Kim J Y, Oh D K, Keasling J D, Kim S W. Increased beta-carotene production in recombinant Escherichia coli harboring an engineered isoprenoid precursor pathway with mevalonate addition. Biotechnol Prog. 2007 May-June; 23(3):599-605.
Yoon, S., Lee, S., Das, A., Ryu, H., Jang, H., Kim, J., Kim., Oh, D., Keasling, J. D., Kim, S. Combinatorial expression of bacterial whole mevalonate pathway for the production of β-carotene in E. coli, Journal of Biotechnology, 2009, 140, 3-4, 218-226.

What is claimed is:

1. A genetically-engineered hydrocarbonoclastic organism that comprises one, or more variant gene(s) encoding one, or more enzymatic protein(s) of the mevalonate synthetic pathway.

2. The genetically-engineered hydrocarbonoclastic organism of claim 1, wherein the organism is a Marinobacter species or a Pseudomonas species of organism.

3. The genetically-engineered organism of claim 2, wherein the organism is Marinobacter atlanticus.

4. The genetically-engineered organism of claim 3, wherein the optimized nucleic acid sequences results in increased biological activity the variant gene(s) relative to the wild-type gene.

5. The genetically-engineered organism of claim 4, wherein the one, or more variant mevalonate pathway gene(s) are selected from the group consisting of: SEQ ID NO: 5; SEQ ID NO: 6; SEQ ID NO: 7; SEQ ID NO: 8; SEQ ID NO: 9; SEQ ID NO: 10; or SEQ ID NO:15.

6. The genetically-engineered organism of claim 5, wherein the organism further comprises one, or more, variant gene(s) encoding one, or more enzymatic proteins of the beta-carotene synthetic pathway.

7. The genetically-engineered organism of claim 6, wherein the variant carotene pathway gene(s) are selected from the group consisting of: SEQ ID NO: 11; SEQ ID NO: 12; SEQ ID NO: 13; SEQ ID NO: 14; SEQ ID NO: 16; SEQ ID NO: 17; SEQ ID NO: 18; SEQ ID NO: 19, SEQ ID NO: 20; SEQ ID NO:21; SEQ ID NO: 27, SEQ ID NO: 28; or SEQ ID NO: 29.

8. The genetically-engineered organism of claim 7, wherein the genetically-engineered organism comprises introduction of a variant blh gene encoding the 15,15′-dioxygenase, (SEQ ID NO: 16) wherein the introduction of the variant blh gene results in the production of retinal and/or retinol.

9. A variant human retinol dehydrogenase 12 (RDH12) gene encoding the retinol dehydrogenase comprising SEQ ID NO: 30.

10. The retinol dehydrogenase gene (RDH12) of claim 9, wherein the nucleic acid sequence is selected from the group consisting of: SEQ ID NO: 17, SEQ ID NO: 18; or SEQ ID NO: 20.

11. The genetically-engineered organism of claim 7, wherein the organism further comprises introduction of a variant RDH12 gene (SEQ ID NO:17, SEQ ID NO: 18 or SEQ ID NO: 20) expressing a variant retinol dehydrogenase 12 (RDH12) SEQ ID NO: 30), wherein the introduction of the variant RDH12 gene results in the conversion of retinal to retinol.

12. The genetically-engineered organism of claim 7, wherein the organism comprises introduction of a variant ybbO gene (SEQ ID NO: 19 or SEQ ID NO: 21), wherein the introduction of the variant ybbO gene results in the conversion of retinal to retinol.

13. The genetically-engineered organism of claim 1, wherein the variant genes of the mevalonate pathway comprise a variant operon of the upper mevalonate pathway comprising SEQ ID NO:1.

14. The genetically-engineered organism of claim 1, wherein the variant gene(s) of the lower mevalonate pathway comprise a variant operon comprising SEQ ID NO: 2.

15. The genetically-engineered organism of claim 6, wherein the variant gene(s) of the beta-carotene pathway comprise a variant operon selected from the group consisting of: SEQ ID NO: 3; SEQ ID NO: 4; SEQ ID NO: 22; SEQ ID NO: 23; SEQ ID NO: 24 or SEQ ID NO: 25.

16. The genetically-engineered organism of claim 6, wherein the variant mevalonate pathway gene(s) comprise the variant operons of SEQ ID NO:1 and SEQ ID NO:2, and the variant carotene pathway gene(s) comprise the variant operons of SEQ ID NO: 3 and SEQ ID NO: 4.

17. The genetically-engineered organism of claim 6, wherein the variant mevalonate pathway gene(s) comprise the variant operons of SEQ ID NO: 1 and SEQ ID NO: 2, and the variant carotene pathway gene(s) comprise the variant operons of SEQ ID NO:22 and SEQ ID NO: 26.

18. An expression vector comprising one, or more, of the variant gene sequences of claim 1.

19. A host cell comprising the expression vector of claim 18.

20. The host cell of claim 19, wherein the host is a hydrocarbononclastic organism.

21. The organism of claim 20, wherein the organism is a Marinobacter species or a Pseudomonas species.

22. The organism of claim 21, wherein the organism is Marinobacter atlanticus.

23. A variant retinol dehydrogenase 12 (RDH12) comprising SEQ ID NO:30.

24. A biofilm comprising a genetically-engineered organism of claim 1.

25. A biofilm bioreactor comprising:
a) the genetically-engineered organisms of claim 1, wherein the organism is supported on a packed bed of particles suitable for the growth and maintenance of a biofilm containing the genetically-engineered organisms;
b) an inlet for the introduction of media and feedstock; and
c) a second inlet for the introduction of extraction solution.

26. The biofilm bioreactor of claim 25, wherein the bioreactor further comprises a mixer or nozzle allowing the introduction of an encapsulant molecule into the bioreactor.

27. The biofilm reactor of claim 25, wherein the extraction solution is a nonpolar solvent.

28. A method of producing an isoprenoid in a biofilm bioreactor using the biofilm bioreactor of claim 25.

29. The method of claim 28, wherein the isoprenoid produced is beta-carotene.

30. The method of claim 28, wherein the isoprenoid produced is retinol.

31. The method of claim 28, wherein the isoprenoid produced is squalane.

32. The method of claim 28, wherein the method comprises the use of an extraction solvent comprising an anti-oxidant or an encapsulant.

33. The method of claim 32, wherein the encapsulant is cyclodextrin.

34. The method of claim 33, wherein the encapsulant molecule forms a liposome.

35. The method of claim 28, wherein the product is a cosmetic ingredient, and the extraction solvent is a component of the cosmetic.

36. The method of claim 35, wherein the cosmetic ingredient is an emollient.

37. The method of claim 36, wherein the emollient is squalane.


SEQUENCE LISTING (39 sequences)

SEQ ID NO: 1 - DNA, 3563 bp, Artificial Sequence
  Description of Artificial Sequence Synthetic polynucleotide
atgaagacagtcgttatcatcgacgctctacgaacccctatcgggaagtacaagggatctctatctcaggtttctgcagtcgatcttggaactcatgttactactcagctccttaagcggcattcgactatctctgaggagatcgaccaggtaatcttcggaaacgtactgcaggcagggaacggtcagaacccagctcgacaaatcgcgatcaactctggactgagtcacgagatccctgcaatgaccgtaaacgaggtctgcggttctggtatgaaggctgttatcctagctaagcagctaatccagctgggtgaagcagaggtgctaatcgcgggtggtatcgagaacatgtctcaggcgccaaagctacagagattcaactacgagactgagtcatacgacgctcctttctctagcatgatgtacgacggtctaactgacgctttcagtggacaggcaatgggattgacagcagaaaacgttgcagaaaagtaccatgttacacgggaggagcaagaccagttctcggtccactcccagctgaaggcggcgcaggcgcaagcagaggggatcttcgcggatgagatcgcacccctagaggtctcaggtactctcgttgagaaggacgaggggatcagaccaaacagtagcgtggagaagctcggaacgctaaagactgtattcaaggaggacggaactgttacagcggggaacgctagtactatcaacgacggagcgtcggcgctcatcatcgcttcgcaagagtacgcggaagcgcacggtctcccctacctggcaatcatcagagacagtgtcgaggtcggtatcgatcctgcatacatggggatcagccccatcaaggctatccaaaagttgctggcacgtaaccaactcacgacagaagagatcgatctttacgagatcaacgaggcgttcgcagctacatctatcgtagtccagcgtgagctcgctctacctgaagagaaggtaaacatctacgggggtggtatcagtttgggacacgcaatcggagcgactggggctaggctactaactagcctttcataccagctaaaccagaaggagaagaagtacggtgtagcttccctatgcatcggtggtgggctaggactcgcaatgcttcttgagcggccccaacagaagaagaactcgcgattctaccagatgagccccgaggaaaggcttgcttccttgctaaacgagggtcaaatctctgcagacactaagaaggagttcgagaacacggctttgtcctcgcagatcgcgaaccacatgatcgagaaccaaatctctgaaacagaggttccaatgggagtggggctgcatctaaccgtagacgagactgactacctagtcccaatggcgaccgaggagccctccgttatcgcagctctctctaacggggcaaagatcgcgcaggggttcaagaccgttaaccagcaaagactgatgcgtggtcaaatcgtattctacgacgtggcggacgcggaatccctcatcgacgaactacaggtcagggaaacagagatcttccagcaggcagagcttagctacccttcaatcgtaaagagaggaggtggactacgggatcttcagtaccgagctttcgacgaatcgttcgtaagtgttgacttcctagttgacgtaaaggatgcgatgggagctaacatcgttaacgcaatgctagagggtgttgcggagctcttccgagagtggttcgcggaacagaagatcctattctctatcttgagtaactacgctacggagtctgtggtaacaatgaagacggctatccccgtttcccggctttcgaagggaagtaacgggagggaaatcgcggagaagatcgtgcttgcttctagatacgcttctctggatccttacagggctgttacgcacaacaagggtatcatgaacgggatcgaggcggttgtattggcaaccgggaacgacactcgggctgtttctgctagttgtcacgcattcgcagtaaaggagggacggtaccagggattgacaagttggactctcgacggggaacagctaatcggagagatctctgtgcccctcgctctggcgacggtagggggtgcgactaaggttctgccaaagtctcaggctgcagcggaccttctagctgtaactgatgcgaaggagctttcgcgtgttgttgcagctgtgggactagctcagaaccttgctgcgctacgggcgttggtcagtgagggaatccagaagggacatatggcgctacaggcacgatcgttggctatgacggttggagctactgggaaggaagtggaggcagtcgcgcagcaactaaagagacagaagactatgaaccaggaccgtgctctagcgatcctgaacgacctgcgaaagcagtaatgacaatcggtatcgacaagatctcgttcttcgttcctccttactacatcgatatgactgctctcgcggaggcacgaaacgtagaccccggtaagttccacatcggaatcggtcaggaccagatggcggttaaccccatcagccaggacatcgttaccttcgctgcgaacgcagctgaggcgatccttactaaggaagacaaggaggcgatcgacatggttatcgttggaactgagagcagcatcgatgaatcaaaggctgcagctgtagttctacatcgacttatggggatccagccattcgctcgaagtttcgagatcaaggagggttgttacggagctacagcgggtctacagcttgctaagaaccacgtagctctacacccagataagaaggttctagtagtcgcagcggatatcgctaagtacggattgaactccgggggtgagccaactcagggagcaggggctgtggcgatgctggtggctagcgaacctagaatcctcgcactaaaggaggataacgttatgttgacgcaagatatctacgacttctggcgtccaacaggtcacccctacccaatggttgacggtccactatccaacgagacttacatccagtctttcgctcaggtatgggacgagcataagaagagaacggggttggatttcgctgactacgacgctcttgcattccacatcccatacaccaagatgggtaagaaggcgttgctggctaagatcagcgaccagacagaggcggaacaggaaagaatcttggcaagatacgaagaatcaatcatctactctagacgagtagggaacctatacactggatctctctacttgggtcttatctcgctactagagaacgctacgacgctgacagcgggaaaccaaatcgggctattcagctacgggtcgggtgcagttgcagagttcttcacaggggagctagtagctggataccaaaaccacctacagaaggaaacgcacttggctctcctagacaacagaaccgagctttccatcgcggaatacgaggctatgttcgctgagactttggacaccgacatcgatcagactcttgaggatgaattgaagtactcgatcagtgctatcaacaacacggtacgttcctaccgaaactga

SEQ ID NO: 2 - DNA, 3387 bp, Artificial Sequence
  Description of Artificial Sequence Synthetic polynucleotide
atgcagacggagcacgtaatcctcctcaacgctcaaggtgtacctacaggaacgttggagaagtacgcggcacacactgctgacacaagactccaccttgctttctcaagttggttgttcaacgcaaagggtcagctactagtcaccagacgagctctatctaagaaggcatggcccggtgtttggactaactctgtctgcggtcacccacagctaggtgaatctaacgaggacgcagtaatcagaaggtgccgctacgagctcggagttgaaatcacacctccagaatctatctaccctgatttccgatacagggcgacagacccaagtggcatcgtagagaacgaggtgtgcccagtcttcgccgctagaacaactagcgccctccagatcaacgacgacgaggtaatggattaccagtggtgcgacctagcagacgttctacacgggatcgatgcgactccatgggccttcagtccttggatggttatgcaagctacgaaccgtgaggcacgtaagcgtctttctgcattcacccagctaaagtaatgatcgcagtaaagacatgcgggaagctttactgggcaggagagtacgcgatcctagaaccagggcagcttgctctcatcaaggacatccccatctacatgagggcggagatcgcattctcggattcttaccgaatctacagtgacatgttcgacttcgctgttgacctacgtcctaaccccgactacagtctcatccaggagacgatcgcattgatgggggatttcttggctgtaagaggacagaacctacgtccattctcactaaagatctgcggaaagatggagcgggagggaaagaagttcggtttgggctcaagcggttctgttgtggtcctagttgtcaaggcattgttggcgctctacaacctaagtgtagaccagaaccttctcttcaagttgactagcgcagtacttcttaagcggggtgacaacggctctatgggggaccttgcatgtatcgttgctgaggatctagtagtttaccaatctttcgacaggcaaaaggcagcggcatggctagaggaggagaacctagcaaccgttctagagagagactgggggttcttcatctcacaggttaagccaactctagagtgcgacttcctagttgggtggactaaggaagtcgcagtaagctcacatatggttcagcagatcaagcagaacatcaaccagaacttcttgtcgtcatccaaggagactgttgtatctttggttgaagctttggagcagggaaaggctgaaaaggttatcgagcaagtagaggtcgctagtaagctactagagggtctttcgactgacatctacacacccctactaaggcagctaaaggaggcgtctcaggaccttcaggctgttgctaagtcatctggcgctggtggcggtgactgcggtatcgctcttagcttcgacgctcagagtacgaagactttgaagaacagatgggcggatctaggaatcgagctcctctaccaggaaaggatcggtcacgatgataagtcttgatggatcgagagcctgtaactgtcagaagttacgctaacatcgcgatcatcaagtactggggtaagaagaaggagaaggaaatggttcctgcgacctcttcaatcagcctcacactagaaaacatgtacaccgagacaactctcagtcccctcccagcgaacgttactgcagatgagttctacatcaacggacagctacaaaacgaagtagaacatgcgaagatgtctaagatcatcgaccggtacagacctgcaggagaggggttcgttcggatcgacacccaaaacaacatgcctacagctgcgggtctatcgtcctcttcgtctggactaagtgctctcgtcaaggcatgcaacgcgtacttcaagctaggactcgatagaagtcaactcgcacaggaggcgaagttcgcaagtggaagctcttctcggagtttctacggaccacttggtgcttgggacaaggactctggtgagatctaccccgttgagaccgatctaaagctagctatgatcatgcttgttctagaggacaagaagaagcctatctcgtcgagagatgggatgaagctctgtgtcgaaactagcactacattcgatgactgggtaaggcagagtgaaaaggactaccaggacatgcttatctacctcaaggaaaacgacttcgcgaagatcggtgagcttacggaaaagaacgcactagcgatgcacgctacaactaagactgcatctcccgcgttcagctaccttactgacgcatcttacgaggctatggacttcgtaagacagttgcgtgagaagggggaggcgtgctacttcactatggacgctggtcccaacgttaaggtattctgccaagagaaggacctcgagcacttgtccgagatcttcggccacaggtacaggctaatcgtttccaagaccaaggacctatcgcaagatgactgctgctaatgactaagaaggtgggagttggacaagcacactcaaagatcatcctcatcggtgaacacgcggtagtatacggatacccagcaatcagtctccctcttctcgaggttgaggtaacttgtaaggttgtgcccgctgaatctccctggcgtctctacgaagaagatactctatctatggcagtatacgcgtcactcgagtacttgaacatcactgaagcttgcatcagatgcgagatcgactcggcgatccctgagaagcgaggtatgggtagttctgcggctatctcaatcgctgcaatccgtgcagtattcgattactaccaggctgaccttcctcatgatgtactagagatccttgtaaacagagcggagatgatcgcacatatgaacccctctggtcttgatgctaagacgtgcctttctgatcaaccaatccggttcatcaagaacgttggattcactgagttggaaatggatctctcggcgtacctcgtaatcgcggacactggcgtatacggtcacactcgtgaggctatccaggtcgtacagaacaaggggaaggacgcgctacccttcctacatgcattgggtgagttgactcagcaggctgaagttgcaatcagtcagaaggacgcggagggacttggtcagatccttagccaggcgcatttgcaccttaaggagatcggagtttcgtctccagaagcggatttcctagtagagactacactctctcacggtgcgcttggcgctaagatgagtggcggcggtctcggaggctgtatcatcgcgctagtcacaaacctcactcatgcacaggagctagcagagcggcttgaggagaagggtgctgtacaaacttggatcgagtctctttga

SEQ ID NO: 3 - DNA, 2911 bp, Artificial Sequence
  Description of Artificial Sequence Synthetic polynucleotide
atggttagtggttcaaaggctggcgtatctccacatagggaaatcgaggtcatgcgacaatcaatcgacgatcacctagcaggcctgttgcccgagactgacagccaggatatcgtctcgttggccatgcgtgagggagttatggcgcctgggaagagaattcggcctttactgatgttgctagctgcacgcgacctcagataccaaggtagtatgcccacattgctcgatctagcctgcgcagtcgagctgacacatactgctagcctaatgctagatgacatgccatgtatggataacgcagagctacgtcgcgggcaacccacaacccacaagaaattcggtgagtctgtagctatcctagcaagcgtcggtctgcttagtaaggcattcggtttaatcgctgccactggtgacctgccaggagagagacgggctcaagccgtaaacgagctaagtactgcagtaggagttcagggtctagtactagggcaattccgagacctaaacgacgctgccctcgatagaacaccagacgcaatcttgagcaccaaccacctaaagacaggcatccttttcagtgctatgctacaaatcgtcgcaatcgcttcagcctcgtctccgtctactcgagagactcttcatgctttcgctctagacttcggccaggctttccaactacttgacgaccttcgagacgatcacccggagacaggtaaagaccgcaacaaagacgctggaaagtccactctagtaaacagacttggtgcggacgctgctagacagaagcttcgagagcatatcgacagtgcagataagcatctcactttcgcatgtccacaaggcggtgccatcagacagttcatgcacttgtggttcggtcaccacttggctgactggtctccggtaatgaagatcgcatgatgggacttatgcttatcgattggtgcgcattggcccttgttgtattcatcggtctacctcacggagcacttgatgcggctatctcattctctatgatctcgtcggcaaagcgaatcgctcgtctagccggaatcctattgatctacctcctactagccactgcattcttccttatctggtaccagctgcctgctttctcgctactaatcttcctcctcatctcaatcatccacttcggcatggcagacttcaacgccagccctagtaagctaaagtggccccacatcatcgctcacggtggggtggtgacggtgtggcttccccttatccaaaagaacgaagtaacgaagcttttctctatccttactaacggtccgactccaatcctttgggacatcctattgatcttcttcttgtgctggtctatcggcgtttgtcttcacacttacgagacattgcgtagcaagcactacaacatcgcattcgagctaatcggtttgatcttcctagcgtggtacgctcctcctctcgttacattcgctacctacttctgcttcatccactctaggagacatttcagtttcgtatggaagcaattgcaacacatgtcaagtaagaagatgatgatcggaagtgcaatcatcctatcttgcacgtcttggctcatcggaggcggaatctacttcttcctaaacagcaagatgatcgctagtgaggctgctctacaaactgtcttcatcggactcgccgctctgaccgtgcctcatatgatcttgatcgatttcatcttccgtcctcattcttcgcggatcaagatcaagaactaatgagagacttgatcctcgtcggcggcggtctagccaacggtcttatcgcctggcgtttgaggcagcgctacccacagctaaacttgcttctgatcgaagctggagaacagcctggaggtaaccatacttggagtttccacgaggacgatttgacacctggacagcatgcttggttggcacctctagtggctcacgcatggccgggttatgaggtgcagttcccagacctccgacgacgtctggcaagaggatactactctatcacttcggagcgcttcgccgaagcactccaccaagcacttggagaaaacatctggctaaactgctcagtgtcagaggttctaccaaatagtgtaaggctcgctaacggggaagctctattagccggggccgttatcgacggtaggggtgtaacggcctcttccgccatgcaaactggttaccagcttttcctaggtcagcaatggaggttgacacagcctcacggccttactgttccgatcctaatggacgcaacggtagcacagcaacagggttacaggtttgtttatacactgccactatcggccgatacgctgctaatcgaggacactaggtacgccaacgttccacaacgtgacgataacgcattgcgtcagacggtgacggactacgcgcactcgaagggatggcagcttgctcagttggagagagaagagacaggctgtctgcctatcacacttgcaggggacatccaggcactatgggcagacgctccaggagtaccgagatccggaatgagggcgggtttgttccatccaactacaggttactcacttccactagccgtggctctagccgacgccatcgctgactcgccaaggctgggaagcgtcccactttaccagctcactaggcaatttgctgagcgccactggcgccgacaaggtttctttcgcctattgaaccgtatgcttttcctagctggacgagaggagaatcgctggcgtgtaatgcagcgtttctacggtcttccagagccaacggtcgagaggttctacgcaggaagacttagtctattcgataaggctaggatccttacaggcaagcctccggtaccactaggagaagcttggagagccgcactaaaccattttcctgacagaagagacaagggatga

SEQ ID NO: 4 - DNA, 3307 bp, Artificial Sequence
  Description of Artificial Sequence Synthetic polynucleotide
atgaagaaaacagttgtcatcggcgcaggcttcgggggacttgcattggctatcagactgcaggccgcgggaatccccacagtcctattggagcagagagacaagccaggagggcgcgcatacgtctggcatgaccagggattcacttttgatgcaggtccgacggtaatcacagatcctactgcccttgaggctcttttcacactagctggtagaagaatggaggactacgtacgtctgttgcctgttaaacccttctaccgtttgtgttgggaaagcggtaagacacttgactacgcaaacgactcggctgagctcgaggctcagatcacccagttcaacccaagagatgtagaaggatacagaaggttccttgcgtactctcaggctgtttttcaggaaggataccttcgactaggatctgttccgtttctatcttttcgagacatgctacgcgctgggccacagttgctaaagctacaggcttggcagtcggtctaccagtcagtttcgaggttcatcgaggatgaacacctcagacaggctttctctttccactcgttactggttggtggtaacccattcactacaagttcgatttacaccctaatccacgccctagagagagagtggggggtttggttccctgagggcggcactggtgccttggtgaacggcatggtcaagctattcacggaccttggcggggaaattgaacttaacgccagagtcgaagaactagtggtagcagataatcgagttagccaagtcaggctggccgacggacgcatcttcgatactgacgctgtcgcttcgaacgcagacgtagtgaacacttacaagaagcttctcggccatcatccggtcggacagaaaagagctgcggcactggaacgtaagtcaatgtcgaactctctattcgtgttgtacttcggtctgaaccaaccacactctcagttggcccaccataccatctgttttggtccacgctaccgagagctaatcgatgagatcttcacaggaagcgctcttgcagacgacttttccctatacttgcattccccatgcgttactgacccatccctagctccaccaggatgtgctagtttctacgtgctagctcctgtgccacacctgggaaacgctccgctggattgggctcaggaaggaccaaagctgcgcgacaggattttcgattacctagaagaacgttacatgccaggcttgcgatctcagttggtaacacagcgtatcttcaccccagcagacttccacgatacgctagacgctcacctagggagtgctttcagcatcgaaccgctgttgacacaaagcgcctggttccgaccacacaaccgagactctgacatcgctaacctgtacctcgtgggtgcgggaactcaccctggggccggcatccctggtgtagtcgcttctgccaaggcaacagcatcattgatgatcgaagacctgcaatgatgagccaacctccgttactcgaccatgcaactcagactatggccaacggatctaagtcgttcgcaacagcagctaagctcttcgacccagctacacgtcgatcggtgctaatgttgtacacctggtgcagacattgtgatgacgtaatcgacgatcagacacacggtttcgcctcagaagccgctgccgaagaggaagctacacagcgactagccaggttgcgcacattgacccttgcagctttcgagggtgctgagatgcaagacccggcattcgcggcctttcaagaggttgccctgacccacggtatcactcctcgtatggccttggatcacctagacggcttcgccatggacgttgcgcaaaccaggtatgttacttttgaagataccctaaggtactgctatcacgtcgccggcgttgtgggtcttatgatggctcgtgtaatgggtgtgcgtgatgaacgtgtactagatagggcttgtgacctaggactagcattccagctgaccaacatcgcacgagacatcatcgacgatgcagcaatcgatcgctgctacctccccgcagagtggctgcaggacgccggactgaccccggagaactatgctgccagggagaaccgtgccgcactagctcgagtagctgagcgcctaatcgacgcagcagagccatactacatctcaagccaggcagggcttcatgacctgccacctcgttgcgcttgggccatcgctacagctcgctcggtctacagggagatcgggatcaaggtaaaggccgctggaggttcggcctgggacagacgtcagcataccagcaagggtgagaagatcgccatgctaatggccgcaccgggacaagttatccgtgcaaaaactacgcgtgttactccacggccagctggtctttggcaacggcctgtctaatggacttccctcagcaacttgaggcttgcgtcaagcaggcgaaccaggctttgtctaggttcatcgcgcctctacccttccagaacacacctgtagtagagaccatgcagtacggtgcactattgggtggaaagcgactaagacctttcttggtttacgctactggtcacatgttcggagtctcaacgaacacgctggatgcacctgcagctgcagtagagtgtatccacgcatactcgctaatccacgacgatctcccggctatggacgacgacgacctacgtcgcggattgcctacttgccacgtaaagttcggcgaggcaaacgcaatcctcgcaggtgacgctctccagacactggccttcagtatcttgtcggatgcggatatgccagaggtatctgatagagatcgaatctctatgatctcggagttggcatctgcgagcggaatcgcagggatgtgcggaggacaggctcttgatcttgacgctgagggtaagcatgttcccctggacgcactcgagaggatccacagacacaagactggtgcactcatccgagctgctgtccgtctcggagcgctcagcgctggggacaagggacgaagggcactgccagtactagacaagtacgcagagtctatcggactagctttccaggtacaggatgatatcctagatgttgttggtgacactgctactctaggtaagaggcagggggcagaccagcagttggggaagagtacttaccccgcacttctagggttggagcaggcgaggaagaaggcgagagacctgatcgacgatgctcgccagagtctaaagcaattggctgagcagtcgttggatacatctgctcttgaggcccttgccgactacatcatccaacgaaacaagtga

SEQ ID NO: 5 - DNA, 2412 bp, Artificial Sequence
  Description of Artificial Sequence Synthetic polynucleotide
atgaagacagtcgttatcatcgacgctctacgaacccctatcgggaagtacaagggatctctatctcaggtttctgcagtcgatcttggaactcatgttactactcagctccttaagcggcattcgactatctctgaggagatcgaccaggtaatcttcggaaacgtactgcaggcagggaacggtcagaacccagctcgacaaatcgcgatcaactctggactgagtcacgagatccctgcaatgaccgtaaacgaggtctgcggttctggtatgaaggctgttatcctagctaagcagctaatccagctgggtgaagcagaggtgctaatcgcgggtggtatcgagaacatgtctcaggcgccaaagctacagagattcaactacgagactgagtcatacgacgctcctttctctagcatgatgtacgacggtctaactgacgctttcagtggacaggcaatgggattgacagcagaaaacgttgcagaaaagtaccatgttacacgggaggagcaagaccagttctcggtccactcccagctgaaggcggcgcaggcgcaagcagaggggatcttcgcggatgagatcgcacccctagaggtctcaggtactctcgttgagaaggacgaggggatcagaccaaacagtagcgtggagaagctcggaacgctaaagactgtattcaaggaggacggaactgttacagcggggaacgctagtactatcaacgacggagcgtcggcgctcatcatcgcttcgcaagagtacgcggaagcgcacggtctcccctacctggcaatcatcagagacagtgtcgaggtcggtatcgatcctgcatacatggggatcagccccatcaaggctatccaaaagttgctggcacgtaaccaactcacgacagaagagatcgatctttacgagatcaacgaggcgttcgcagctacatctatcgtagtccagcgtgagctcgctctacctgaagagaaggtaaacatctacgggggtggtatcagtttgggacacgcaatcggagcgactggggctaggctactaactagcctttcataccagctaaaccagaaggagaagaagtacggtgtagcttccctatgcatcggtggtgggctaggactcgcaatgcttcttgagcggccccaacagaagaagaactcgcgattctaccagatgagccccgaggaaaggcttgcttccttgctaaacgagggtcaaatctctgcagacactaagaaggagttcgagaacacggctttgtcctcgcagatcgcgaaccacatgatcgagaaccaaatctctgaaacagaggttccaatgggagtggggctgcatctaaccgtagacgagactgactacctagtcccaatggcgaccgaggagccctccgttatcgcagctctctctaacggggcaaagatcgcgcaggggttcaagaccgttaaccagcaaagactgatgcgtggtcaaatcgtattctacgacgtggcggacgcggaatccctcatcgacgaactacaggtcagggaaacagagatcttccagcaggcagagcttagctacccttcaatcgtaaagagaggaggtggactacgggatcttcagtaccgagctttcgacgaatcgttcgtaagtgttgacttcctagttgacgtaaaggatgcgatgggagctaacatcgttaacgcaatgctagagggtgttgcggagctcttccgagagtggttcgcggaacagaagatcctattctctatcttgagtaactacgctacggagtctgtggtaacaatgaagacggctatccccgtttcccggctttcgaagggaagtaacgggagggaaatcgcggagaagatcgtgcttgcttctagatacgcttctctggatccttacagggctgttacgcacaacaagggtatcatgaacgggatcgaggcggttgtattggcaaccgggaacgacactcgggctgtttctgctagttgtcacgcattcgcagtaaaggagggacggtaccagggattgacaagttggactctcgacggggaacagctaatcggagagatctctgtgcccctcgctctggcgacggtagggggtgcgactaaggttctgccaaagtctcaggctgcagcggaccttctagctgtaactgatgcgaaggagctttcgcgtgttgttgcagctgtgggactagctcagaaccttgctgcgctacgggcgttggtcagtgagggaatccagaagggacatatggcgctacaggcacgatcgttggctatgacggttggagctactgggaaggaagtggaggcagtcgcgcagcaactaaagagacagaagactatgaaccaggaccgtgctctagcgatcctgaacgacctgcgaaagcagtga

SEQ ID NO: 6 - DNA, 1152 bp, Artificial Sequence
  Description of Artificial Sequence Synthetic polynucleotide
atgacaatcggtatcgacaagatctcgttcttcgttcctccttactacatcgatatgactgctctcgcggaggcacgaaacgtagaccccggtaagttccacatcggaatcggtcaggaccagatggcggttaaccccatcagccaggacatcgttaccttcgctgcgaacgcagctgaggcgatccttactaaggaagacaaggaggcgatcgacatggttatcgttggaactgagagcagcatcgatgaatcaaaggctgcagctgtagttctacatcgacttatggggatccagccattcgctcgaagtttcgagatcaaggagggttgttacggagctacagcgggtctacagcttgctaagaaccacgtagctctacacccagataagaaggttctagtagtcgcagcggatatcgctaagtacggattgaactccgggggtgagccaactcagggagcaggggctgtggcgatgctggtggctagcgaacctagaatcctcgcactaaaggaggataacgttatgttgacgcaagatatctacgacttctggcgtccaacaggtcacccctacccaatggttgacggtccactatccaacgagacttacatccagtctttcgctcaggtatgggacgagcataagaagagaacggggttggatttcgctgactacgacgctcttgcattccacatcccatacaccaagatgggtaagaaggcgttgctggctaagatcagcgaccagacagaggcggaacaggaaagaatcttggcaagatacgaagaatcaatcatctactctagacgagtagggaacctatacactggatctctctacttgggtcttatctcgctactagagaacgctacgacgctgacagcgggaaaccaaatcgggctattcagctacgggtcgggtgcagttgcagagttcttcacaggggagctagtagctggataccaaaaccacctacagaaggaaacgcacttggctctcctagacaacagaaccgagctttccatcgcggaatacgaggctatgttcgctgagactttggacaccgacatcgatcagactcttgaggatgaattgaagtactcgatcagtgctatcaacaacacggtacgttcctaccgaaactga

SEQ ID NO: 7 - DNA, 879 bp, Artificial Sequence
  Description of Artificial Sequence Synthetic polynucleotide
atgactaagaaggtgggagttggacaagcacactcaaagatcatcctcatcggtgaacacgcggtagtatacggatacccagcaatcagtctccctcttctcgaggttgaggtaacttgtaaggttgtgcccgctgaatctccctggcgtctctacgaagaagatactctatctatggcagtatacgcgtcactcgagtacttgaacatcactgaagcttgcatcagatgcgagatcgactcggcgatccctgagaagcgaggtatgggtagttctgcggctatctcaatcgctgcaatccgtgcagtattcgattactaccaggctgaccttcctcatgatgtactagagatccttgtaaacagagcggagatgatcgcacatatgaacccctctggtcttgatgctaagacgtgcctttctgatcaaccaatccggttcatcaagaacgttggattcactgagttggaaatggatctctcggcgtacctcgtaatcgcggacactggcgtatacggtcacactcgtgaggctatccaggtcgtacagaacaaggggaaggacgcgctacccttcctacatgcattgggtgagttgactcagcaggctgaagttgcaatcagtcagaaggacgcggagggacttggtcagatccttagccaggcgcatttgcaccttaaggagatcggagtttcgtctccagaagcggatttcctagtagagactacactctctcacggtgcgcttggcgctaagatgagtggcggcggtctcggaggctgtatcatcgcgctagtcacaaacctcactcatgcacaggagctagcagagcggcttgaggagaagggtgctgtacaaacttggatcgagtctctttga

SEQ ID NO: 8 - DNA, 1008 bp, Artificial Sequence
  Description of Artificial Sequence Synthetic polynucleotide
atgatcgcagtaaagacatgcgggaagctttactgggcaggagagtacgcgatcctagaaccagggcagcttgctctcatcaaggacatccccatctacatgagggcggagatcgcattctcggattcttaccgaatctacagtgacatgttcgacttcgctgttgacctacgtcctaaccccgactacagtctcatccaggagacgatcgcattgatgggggatttcttggctgtaagaggacagaacctacgtccattctcactaaagatctgcggaaagatggagcgggagggaaagaagttcggtttgggctcaagcggttctgttgtggtcctagttgtcaaggcattgttggcgctctacaacctaagtgtagaccagaaccttctcttcaagttgactagcgcagtacttcttaagcggggtgacaacggctctatgggggaccttgcatgtatcgttgctgaggatctagtagtttaccaatctttcgacaggcaaaaggcagcggcatggctagaggaggagaacctagcaaccgttctagagagagactgggggttcttcatctcacaggttaagccaactctagagtgcgacttcctagttgggtggactaaggaagtcgcagtaagctcacatatggttcagcagatcaagcagaacatcaaccagaacttcttgtcgtcatccaaggagactgttgtatctttggttgaagctttggagcagggaaaggctgaaaaggttatcgagcaagtagaggtcgctagtaagctactagagggtctttcgactgacatctacacacccctactaaggcagctaaaggaggcgtctcaggaccttcaggctgttgctaagtcatctggcgctggtggcggtgactgcggtatcgctcttagcttcgacgctcagagtacgaagactttgaagaacagatgggcggatctaggaatcgagctcctctaccaggaaaggatcggtcacgatgataagtcttga

SEQ ID NO: 9 - DNA, 954 bp, Artificial Sequence
  Description of Artificial Sequence Synthetic polynucleotide
atggatcgagagcctgtaactgtcagaagttacgctaacatcgcgatcatcaagtactggggtaagaagaaggagaaggaaatggttcctgcgacctcttcaatcagcctcacactagaaaacatgtacaccgagacaactctcagtcccctcccagcgaacgttactgcagatgagttctacatcaacggacagctacaaaacgaagtagaacatgcgaagatgtctaagatcatcgaccggtacagacctgcaggagaggggttcgttcggatcgacacccaaaacaacatgcctacagctgcgggtctatcgtcctcttcgtctggactaagtgctctcgtcaaggcatgcaacgcgtacttcaagctaggactcgatagaagtcaactcgcacaggaggcgaagttcgcaagtggaagctcttctcggagtttctacggaccacttggtgcttgggacaaggactctggtgagatctaccccgttgagaccgatctaaagctagctatgatcatgcttgttctagaggacaagaagaagcctatctcgtcgagagatgggatgaagctctgtgtcgaaactagcactacattcgatgactgggtaaggcagagtgaaaaggactaccaggacatgcttatctacctcaaggaaaacgacttcgcgaagatcggtgagcttacggaaaagaacgcactagcgatgcacgctacaactaagactgcatctcccgcgttcagctaccttactgacgcatcttacgaggctatggacttcgtaagacagttgcgtgagaagggggaggcgtgctacttcactatggacgctggtcccaacgttaaggtattctgccaagagaaggacctcgagcacttgtccgagatcttcggccacaggtacaggctaatcgtttccaagaccaaggacctatcgcaagatgactgctgctga

SEQ ID NO: 10 - DNA, 549 bp, Artificial Sequence
  Description of Artificial Sequence Synthetic polynucleotide
atgcagacggagcacgtaatcctcctcaacgctcaaggtgtacctacaggaacgttggagaagtacgcggcacacactgctgacacaagactccaccttgctttctcaagttggttgttcaacgcaaagggtcagctactagtcaccagacgagctctatctaagaaggcatggcccggtgtttggactaactctgtctgcggtcacccacagctaggtgaatctaacgaggacgcagtaatcagaaggtgccgctacgagctcggagttgaaatcacacctccagaatctatctaccctgatttccgatacagggcgacagacccaagtggcatcgtagagaacgaggtgtgcccagtcttcgccgctagaacaactagcgccctccagatcaacgacgacgaggtaatggattaccagtggtgcgacctagcagacgttctacacgggatcgatgcgactccatgggccttcagtccttggatggttatgcaagctacgaaccgtgaggcacgtaagcgtctttctgcattcacccagctaaagtga

SEQ ID NO: 11 - DNA, 924 bp, Artificial Sequence
  Description of Artificial Sequence Synthetic polynucleotide
atggttagtggttcaaaggctggcgtatctccacatagggaaatcgaggtcatgcgacaatcaatcgacgatcacctagcaggcctgttgcccgagactgacagccaggatatcgtctcgttggccatgcgtgagggagttatggcgcctgggaagagaattcggcctttactgatgttgctagctgcacgcgacctcagataccaaggtagtatgcccacattgctcgatctagcctgcgcagtcgagctgacacatactgctagcctaatgctagatgacatgccatgtatggataacgcagagctacgtcgcgggcaacccacaacccacaagaaattcggtgagtctgtagctatcctagcaagcgtcggtctgcttagtaaggcattcggtttaatcgctgccactggtgacctgccaggagagagacgggctcaagccgtaaacgagctaagtactgcagtaggagttcagggtctagtactagggcaattccgagacctaaacgacgctgccctcgatagaacaccagacgcaatcttgagcaccaaccacctaaagacaggcatccttttcagtgctatgctacaaatcgtcgcaatcgcttcagcctcgtctccgtctactcgagagactcttcatgctttcgctctagacttcggccaggctttccaactacttgacgaccttcgagacgatcacccggagacaggtaaagaccgcaacaaagacgctggaaagtccactctagtaaacagacttggtgcggacgctgctagacagaagcttcgagagcatatcgacagtgcagataagcatctcactttcgcatgtccacaaggcggtgccatcagacagttcatgcacttgtggttcggtcaccacttggctgactggtctccggtaatgaagatcgcatga

SEQ ID NO: 12 - DNA, 930 bp, Artificial Sequence
  Description of Artificial Sequence Synthetic polynucleotide
atgagccaacctccgttactcgaccatgcaactcagactatggccaacggatctaagtcgttcgcaacagcagctaagctcttcgacccagctacacgtcgatcggtgctaatgttgtacacctggtgcagacattgtgatgacgtaatcgacgatcagacacacggtttcgcctcagaagccgctgccgaagaggaagctacacagcgactagccaggttgcgcacattgacccttgcagctttcgagggtgctgagatgcaagacccggcattcgcggcctttcaagaggttgccctgacccacggtatcactcctcgtatggccttggatcacctagacggcttcgccatggacgttgcgcaaaccaggtatgttacttttgaagataccctaaggtactgctatcacgtcgccggcgttgtgggtcttatgatggctcgtgtaatgggtgtgcgtgatgaacgtgtactagatagggcttgtgacctaggactagcattccagctgaccaacatcgcacgagacatcatcgacgatgcagcaatcgatcgctgctacctccccgcagagtggctgcaggacgccggactgaccccggagaactatgctgccagggagaaccgtgccgcactagctcgagtagctgagcgcctaatcgacgcagcagagccatactacatctcaagccaggcagggcttcatgacctgccacctcgttgcgcttgggccatcgctacagctcgctcggtctacagggagatcgggatcaaggtaaaggccgctggaggttcggcctgggacagacgtcagcataccagcaagggtgagaagatcgccatgctaatggccgcaccgggacaagttatccgtgcaaaaactacgcgtgttactccacggccagctggtctttggcaacggcctgtctga

SEQ ID NO: 13 - DNA, 1479 bp, Artificial Sequence
  Description of Artificial Sequence Synthetic polynucleotide
atgaagaaaacagttgtcatcggcgcaggcttcgggggacttgcattggctatcagactgcaggccgcgggaatccccacagtcctattggagcagagagacaagccaggagggcgcgcatacgtctggcatgaccagggattcacttttgatgcaggtccgacggtaatcacagatcctactgcccttgaggctcttttcacactagctggtagaagaatggaggactacgtacgtctgttgcctgttaaacccttctaccgtttgtgttgggaaagcggtaagacacttgactacgcaaacgactcggctgagctcgaggctcagatcacccagttcaacccaagagatgtagaaggatacagaaggttccttgcgtactctcaggctgtttttcaggaaggataccttcgactaggatctgttccgtttctatcttttcgagacatgctacgcgctgggccacagttgctaaagctacaggcttggcagtcggtctaccagtcagtttcgaggttcatcgaggatgaacacctcagacaggctttctctttccactcgttactggttggtggtaacccattcactacaagttcgatttacaccctaatccacgccctagagagagagtggggggtttggttccctgagggcggcactggtgccttggtgaacggcatggtcaagctattcacggaccttggcggggaaattgaacttaacgccagagtcgaagaactagtggtagcagataatcgagttagccaagtcaggctggccgacggacgcatcttcgatactgacgctgtcgcttcgaacgcagacgtagtgaacacttacaagaagcttctcggccatcatccggtcggacagaaaagagctgcggcactggaacgtaagtcaatgtcgaactctctattcgtgttgtacttcggtctgaaccaaccacactctcagttggcccaccataccatctgttttggtccacgctaccgagagctaatcgatgagatcttcacaggaagcgctcttgcagacgacttttccctatacttgcattccccatgcgttactgacccatccctagctccaccaggatgtgctagtttctacgtgctagctcctgtgccacacctgggaaacgctccgctggattgggctcaggaaggaccaaagctgcgcgacaggattttcgattacctagaagaacgttacatgccaggcttgcgatctcagttggtaacacagcgtatcttcaccccagcagacttccacgatacgctagacgctcacctagggagtgctttcagcatcgaaccgctgttgacacaaagcgcctggttccgaccacacaaccgagactctgacatcgctaacctgtacctcgtgggtgcgggaactcaccctggggccggcatccctggtgtagtcgcttctgccaaggcaacagcatcattgatgatcgaagacctgcaatga

SEQ ID NO: 14 - DNA, 1161 bp, Artificial Sequence
  Description of Artificial Sequence Synthetic polynucleotide
atgagagacttgatcctcgtcggcggcggtctagccaacggtcttatcgcctggcgtttgaggcagcgctacccacagctaaacttgcttctgatcgaagctggagaacagcctggaggtaaccatacttggagtttccacgaggacgatttgacacctggacagcatgcttggttggcacctctagtggctcacgcatggccgggttatgaggtgcagttcccagacctccgacgacgtctggcaagaggatactactctatcacttcggagcgcttcgccgaagcactccaccaagcacttggagaaaacatctggctaaactgctcagtgtcagaggttctaccaaatagtgtaaggctcgctaacggggaagctctattagccggggccgttatcgacggtaggggtgtaacggcctcttccgccatgcaaactggttaccagcttttcctaggtcagcaatggaggttgacacagcctcacggccttactgttccgatcctaatggacgcaacggtagcacagcaacagggttacaggtttgtttatacactgccactatcggccgatacgctgctaatcgaggacactaggtacgccaacgttccacaacgtgacgataacgcattgcgtcagacggtgacggactacgcgcactcgaagggatggcagcttgctcagttggagagagaagagacaggctgtctgcctatcacacttgcaggggacatccaggcactatgggcagacgctccaggagtaccgagatccggaatgagggcgggtttgttccatccaactacaggttactcacttccactagccgtggctctagccgacgccatcgctgactcgccaaggctgggaagcgtcccactttaccagctcactaggcaatttgctgagcgccactggcgccgacaaggtttctttcgcctattgaaccgtatgcttttcctagctggacgagaggagaatcgctggcgtgtaatgcagcgtttctacggtcttccagagccaacggtcgagaggttctacgcaggaagacttagtctattcgataaggctaggatccttacaggcaagcctccggtaccactaggagaagcttggagagccgcactaaaccattttcctgacagaagagacaagggatga

SEQ ID NO: 15 - DNA, 900 bp, Artificial Sequence
  Description of Artificial Sequence Synthetic polynucleotide
atggacttccctcagcaacttgaggcttgcgtcaagcaggcgaaccaggctttgtctaggttcatcgcgcctctacccttccagaacacacctgtagtagagaccatgcagtacggtgcactattgggtggaaagcgactaagacctttcttggtttacgctactggtcacatgttcggagtctcaacgaacacgctggatgcacctgcagctgcagtagagtgtatccacgcatactcgctaatccacgacgatctcccggctatggacgacgacgacctacgtcgcggattgcctacttgccacgtaaagttcggcgaggcaaacgcaatcctcgcaggtgacgctctccagacactggccttcagtatcttgtcggatgcggatatgccagaggtatctgatagagatcgaatctctatgatctcggagttggcatctgcgagcggaatcgcagggatgtgcggaggacaggctcttgatcttgacgctgagggtaagcatgttcccctggacgcactcgagaggatccacagacacaagactggtgcactcatccgagctgctgtccgtctcggagcgctcagcgctggggacaagggacgaagggcactgccagtactagacaagtacgcagagtctatcggactagctttccaggtacaggatgatatcctagatgttgttggtgacactgctactctaggtaagaggcagggggcagaccagcagttggggaagagtacttaccccgcacttctagggttggagcaggcgaggaagaaggcgagagacctgatcgacgatgctcgccagagtctaaagcaattggctgagcagtcgttggatacatctgctcttgaggcccttgccgactacatcatccaacgaaacaagtga

SEQ ID NO: 16 - DNA, 828 bp, Artificial Sequence
  Description of Artificial Sequence Synthetic polynucleotide
atgggacttatgcttatcgattggtgcgcattggcccttgttgtattcatcggtctacctcacggagcacttgatgcggctatctcattctctatgatctcgtcggcaaagcgaatcgctcgtctagccggaatcctattgatctacctcctactagccactgcattcttccttatctggtaccagctgcctgctttctcgctactaatcttcctcctcatctcaatcatccacttcggcatggcagacttcaacgccagccctagtaagctaaagtggccccacatcatcgctcacggtggggtggtgacggtgtggcttccccttatccaaaagaacgaagtaacgaagcttttctctatccttactaacggtccgactccaatcctttgggacatcctattgatcttcttcttgtgctggtctatcggcgtttgtcttcacacttacgagacattgcgtagcaagcactacaacatcgcattcgagctaatcggtttgatcttcctagcgtggtacgctcctcctctcgttacattcgctacctacttctgcttcatccactctaggagacatttcagtttcgtatggaagcaattgcaacacatgtcaagtaagaagatgatgatcggaagtgcaatcatcctatcttgcacgtcttggctcatcggaggcggaatctacttcttcctaaacagcaagatgatcgctagtgaggctgctctacaaactgtcttcatcggactcgccgctctgaccgtgcctcatatgatcttgatcgatttcatcttccgtcctcattcttcgcggatcaagatcaagaactga

SEQ ID NO: 17 - DNA, 951 bp, Artificial Sequence
  Description of Artificial Sequence Synthetic polynucleotide
atgctggtcacactagggctactaacatcattcttctcgttcctgtacatggttgcgccttctattaggaagttctttgcaggaggagtatgccgaactaacgtgcagctacctggaaaggtcgtggttatcactggggcgaacactgggatcggtaaggagacggcacgtgagctcgcgtcgcgtggggctcgtgtatacatcgcatgcagggacgtcctgaagggtgagtctgctgcatccgagatccgagtagacaccaagaactcgcaggtgctagtacgaaagctcgacttgagcgacactaagagtatcagggcgttcgctgaaggtttcctggcagaggagaagcaacttcacatcctgatcaataacgcaggtgttatgatgtgcccgtactcgaaaacagcggacggattcgagacccatctaggtgtaaaccaccttggacacttcttgctgacttacttgctactagagcgactcaaggtttctgcgccagcaagggtcgtcaacgtatcgtcggtagcgcaccatatcgggaagatcccatttcacgacctacagtcggagaagagatactcgcgaggtttcgcttactgccactcgaagctagctaacgttctcttcactcgggaattggctaagcgtttgcaagggacaggtgtaacgacatacgcggttcaccctggggttgtcaggtctgagcttgttagacattcgtctcttctttgtctactatggcgtctattcagtcctttcgtgaagacagctcgggagggggctcagactagcttgcactgtgctctggcggaagggctagaacctctatctggaaagtacttcagtgattgcaagcggacctgggtgagtccaagagcgagaaacaacaagactgctgagagattgtggaatgtcagctgcgagctacttggaatccgatgggagtga

SEQ ID NO: 18 - DNA, 873 bp, Artificial Sequence
  Description of Artificial Sequence Synthetic polynucleotide
gcaggaggagtatgccgaactaacgtgcagctacctggaaaggtcgtggttatcactggggcgaacactgggatcggtaaggagacggcacgtgagctcgcgtcgcgtggggctcgtgtatacatcgcatgcagggacgtcctgaagggtgagtctgctgcatccgagatccgagtagacaccaagaactcgcaggtgctagtacgaaagctcgacttgagcgacactaagagtatcagggcgttcgctgaaggtttcctggcagaggagaagcaacttcacatcctgatcaataacgcaggtgttatgatgtgcccgtactcgaaaacagcggacggattcgagacccatctaggtgtaaaccaccttggacacttcttgctgacttacttgctactagagcgactcaaggtttctgcgccagcaagggtcgtcaacgtatcgtcggtagcgcaccatatcgggaagatcccatttcacgacctacagtcggagaagagatactcgcgaggtttcgcttactgccactcgaagctagctaacgttctcttcactcgggaattggctaagcgtttgcaagggacaggtgtaacgacatacgcggttcaccctggggttgtcaggtctgagcttgttagacattcgtctcttctttgtctactatggcgtctattcagtcctttcgtgaagacagctcgggagggggctcagactagcttgcactgtgctctggcggaagggctagaacctctatctggaaagtacttcagtgattgcaagcggacctgggtgagtccaagagcgagaaacaacaagactgctgagagattgtggaatgtcagctgcgagctacttggaatccgatgggagtga

SEQ ID NO: 19 - DNA, 810 bp, Artificial Sequence
  Description of Artificial Sequence Synthetic polynucleotide
atgactcacaaggctacagagatcctaacagggaaggttatgcaaaagagtgtcctaatcactggttgcagttctggtatcggcctagagtcggctctagaactcaagagacagggtttccacgtgctggcgggatgcaggaagccagatgacgtcgaacgcatgaacagcatgggtttcactggagtattgatcgaccttgacagccccgagagtgtcgacagagcagcagatgaggttatcgcacttacagacaactgtctatacggtatcttcaacaacgcaggtttcggaatgtacggacctctatcgactatcagtcgagcacagatggaacagcagttcagtgcgaacttcttcggcgcgcaccagttgacaatgagacttcttcccgctatgttgccgcacggggaaggtcgcatcgtaatgacgtcttctgtaatgggactaatctcgacgcctggtagaggtgcgtacgcagcttcgaagtacgccttggaggcatggagcgacgcgctgcgtatggaacttaggcactcggggatcaaggttagcctgatcgagcctgggccaatccgcactcgattcaccgacaacgttaaccaaactcaatcagacaagcccgtcgaaaaccccggtatcgcggcacgattcacgcttggacctgaggccgtagttgacaaggttcgtcacgcattcatcagtgagaagccgaagatgcgatacccagtaactctggtaacctgggctgttatggttctaaagcgcctgctgccgggaagggtaatggataagatcctacagggatga

SEQ ID NO: 20 - DNA, 912 bp, Artificial Sequence
  Description of Artificial Sequence Synthetic polynucleotide
atggcaggaggagtatgccgaactaacgtgcagctacctggaaaggtcgtggttatcactggggcgaacactgggatcggtaaggagacggcacgtgagctcgcgtcgcgtggggctcgtgtatacatcgcatgcagggacgtcctgaagggtgagtctgctgcatccgagatccgagtagacaccaagaactcgcaggtgctagtacgaaagctcgacttgagcgacactaagagtatcagggcgttcgctgaaggtttcctggcagaggagaagcaacttcacatcctgatcaataacgcaggtgttatgatgtgcccgtactcgaaaacagcggacggattcgagacccatctaggtgtaaaccaccttggacacttcttgctgacttacttgctactagagcgactcaaggtttctgcgccagcaagggtcgtcaacgtatcgtcggtagcgcaccatatcgggaagatcccatttcacgacctacagtcggagaagagatactcgcgaggtttcgcttactgccactcgaagctagctaacgttctcttcactcgggaattggctaagcgtttgcaagggacaggtgtaacgacatacgcggttcaccctggggttgtcaggtctgagcttgttagacattcgtctcttctttgtctactatggcgtctattcagtcctttcgtgaagacagctcgggagggggctcagactagcttgcactgtgctctggcggaagggctagaacctctatctggaaagtacttcagtgattgcaagcggacctgggtgagtccaagagcgagaaacaacaagactgctgagagattgtggaatgtcagctgcgagctacttggaatccgatgggagcttaccgccgcactcgagcatcaccatcaccatcactga

SEQ ID NO: 21 - DNA, 846 bp, Artificial Sequence
  Description of Artificial Sequence Synthetic polynucleotide
atgactcacaaggctacagagatcctaacagggaaggttatgcaaaagagtgtcctaatcactggttgcagttctggtatcggcctagagtcggctctagaactcaagagacagggtttccacgtgctggcgggatgcaggaagccagatgacgtcgaacgcatgaacagcatgggtttcactggagtattgatcgaccttgacagccccgagagtgtcgacagagcagcagatgaggttatcgcacttacagacaactgtctatacggtatcttcaacaacgcaggtttcggaatgtacggacctctatcgactatcagtcgagcacagatggaacagcagttcagtgcgaacttcttcggcgcgcaccagttgacaatgagacttcttcccgctatgttgccgcacggggaaggtcgcatcgtaatgacgtcttctgtaatgggactaatctcgacgcctggtagaggtgcgtacgcagcttcgaagtacgccttggaggcatggagcgacgcgctgcgtatggaacttaggcactcggggatcaaggttagcctgatcgagcctgggccaatccgcactcgattcaccgacaacgttaaccaaactcaatcagacaagcccgtcgaaaaccccggtatcgcggcacgattcacgcttggacctgaggccgtagttgacaaggttcgtcacgcattcatcagtgagaagccgaagatgcgatacccagtaactctggtaacctgggctgttatggttctaaagcgcctgctgccgggaagggtaatggataagatcctacagggacttaccgccgcactcgagcatcaccatcaccatcactga

SEQ ID NO: 22 - DNA, 3786 bp, Artificial Sequence
  Description of Artificial Sequence Synthetic polynucleotide
atggttagtggttcaaaggctggcgtatctccacatagggaaatcgaggtcatgcgacaatcaatcgacgatcacctagcaggcctgttgcccgagactgacagccaggatatcgtctcgttggccatgcgtgagggagttatggcgcctgggaagagaattcggcctttactgatgttgctagctgcacgcgacctcagataccaaggtagtatgcccacattgctcgatctagcctgcgcagtcgagctgacacatactgctagcctaatgctagatgacatgccatgtatggataacgcagagctacgtcgcgggcaacccacaacccacaagaaattcggtgagtctgtagctatcctagcaagcgtcggtctgcttagtaaggcattcggtttaatcgctgccactggtgacctgccaggagagagacgggctcaagccgtaaacgagctaagtactgcagtaggagttcagggtctagtactagggcaattccgagacctaaacgacgctgccctcgatagaacaccagacgcaatcttgagcaccaaccacctaaagacaggcatccttttcagtgctatgctacaaatcgtcgcaatcgcttcagcctcgtctccgtctactcgagagactcttcatgctttcgctctagacttcggccaggctttccaactacttgacgaccttcgagacgatcacccggagacaggtaaagaccgcaacaaagacgctggaaagtccactctagtaaacagacttggtgcggacgctgctagacagaagcttcgagagcatatcgacagtgcagataagcatctcactttcgcatgtccacaaggcggtgccatcagacagttcatgcacttgtggttcggtcaccacttggctgactggtctccggtaatgaagatcgcttgatgggacttatgcttatcgattggtgcgcattggcccttgttgtattcatcggtctacctcacggagcacttgatgcggctatctcattctctatgatctcgtcggcaaagcgaatcgctcgtctagccggaatcctattgatctacctcctactagccactgcattcttccttatctggtaccagctgcctgctttctcgctactaatcttcctcctcatctcaatcatccacttcggcatggcagacttcaacgccagccctagtaagctaaagtggccccacatcatcgctcacggtggggtggtgacggtgtggcttccccttatccaaaagaacgaagtaacgaagcttttctctatccttactaacggtccgactccaatcctttgggacatcctattgatcttcttcttgtgctggtctatcggcgtttgtcttcacacttacgagacattgcgtagcaagcactacaacatcgcattcgagctaatcggtttgatcttcctagcgtggtacgctcctcctctcgttacattcgctacctacttctgcttcatccactctaggagacatttcagtttcgtatggaagcaattgcaacacatgtcaagtaagaagatgatgatcggaagtgcaatcatcctatcttgcacgtcttggctcatcggaggcggaatctacttcttcctaaacagcaagatgatcgctagtgaggctgctctacaaactgtcttcatcggactcgccgctctgaccgtgcctcatatgatcttgatcgatttcatcttccgtcctcattcttcgcggatcaagatcaagaactaatgagagacttgatcctcgtcggcggcggtctagccaacggtcttatcgcctggcgtttgaggcagcgctacccacagctaaacttgcttctgatcgaagctggagaacagcctggaggtaaccatacttggagtttccacgaggacgatttgacacctggacagcatgcttggttggcacctctagtggctcacgcatggccgggttatgaggtgcagttcccagacctccgacgacgtctggcaagaggatactactctatcacttcggagcgcttcgccgaagcactccaccaagcacttggagaaaacatctggctaaactgctcagtgtcagaggttctaccaaatagtgtaaggctcgctaacggggaagctctattagccggggccgttatcgacggtaggggtgtaacggcctcttccgccatgcaaactggttaccagcttttcctaggtcagcaatggaggttgacacagcctcacggccttactgttccgatcctaatggacgcaacggtagcacagcaacagggttacaggtttgtttatacactgccactatcggccgatacgctgctaatcgaggacactaggtacgccaacgttccacaacgtgacgataacgcattgcgtcagacggtgacggactacgcgcactcgaagggatggcagcttgctcagttggagagagaagagacaggctgtctgcctatcacacttgcaggggacatccaggcactatgggcagacgctccaggagtaccgagatccggaatgagggcgggtttgttccatccaactacaggttactcacttccactagccgtggctctagccgacgccatcgctgactcgccaaggctgggaagcgtcccactttaccagctcactaggcaatttgctgagcgccactggcgccgacaaggtttctttcgcctattgaaccgtatgcttttcctagctggacgagaggagaatcgctggcgtgtaatgcagcgtttctacggtcttccagagccaacggtcgagaggttctacgcaggaagacttagtctattcgataaggctaggatccttacaggcaagcctccggtaccactaggagaagcttggagagccgcactaaaccattttcctgacagaagagacaaggggtgatggcaggaggagtatgccgaactaacgtgcagctacctggaaaggtcgtggttatcactggggcgaacactgggatcggtaaggagacggcacgtgagctcgcgtcgcgtggggctcgtgtatacatcgcatgcagggacgtcctgaagggtgagtctgctgcatccgagatccgagtagacaccaagaactcgcaggtgctagtacgaaagctcgacttgagcgacactaagagtatcagggcgttcgctgaaggtttcctggcagaggagaagcaacttcacatcctgatcaataacgcaggtgttatgatgtgcccgtactcgaaaacagcggacggattcgagacccatctaggtgtaaaccaccttggacacttcttgctgacttacttgctactagagcgactcaaggtttctgcgccagcaagggtcgtcaacgtatcgtcggtagcgcaccatatcgggaagatcccatttcacgacctacagtcggagaagagatactcgcgaggtttcgcttactgccactcgaagctagctaacgttctcttcactcgggaattggctaagcgtttgcaagggacaggtgtaacgacatacgcggttcaccctggggttgtcaggtctgagcttgttagacattcgtctcttctttgtctactatggcgtctattcagtcctttcgtgaagacagctcgggagggggctcagactagcttgcactgtgctctggcggaagggctagaacctctatctggaaagtacttcagtgattgcaagcggacctgggtgagtccaagagcgagaaacaacaagactgctgagagattgtggaatgtcagctgcgagctacttggaatccgatgggagtga

SEQ ID NO: 23 - DNA, 3822 bp, Artificial Sequence
  Description of Artificial Sequence Synthetic polynucleotide
atggttagtggttcaaaggctggcgtatctccacatagggaaatcgaggtcatgcgacaatcaatcgacgatcacctagcaggcctgttgcccgagactgacagccaggatatcgtctcgttggccatgcgtgagggagttatggcgcctgggaagagaattcggcctttactgatgttgctagctgcacgcgacctcagataccaaggtagtatgcccacattgctcgatctagcctgcgcagtcgagctgacacatactgctagcctaatgctagatgacatgccatgtatggataacgcagagctacgtcgcgggcaacccacaacccacaagaaattcggtgagtctgtagctatcctagcaagcgtcggtctgcttagtaaggcattcggtttaatcgctgccactggtgacctgccaggagagagacgggctcaagccgtaaacgagctaagtactgcagtaggagttcagggtctagtactagggcaattccgagacctaaacgacgctgccctcgatagaacaccagacgcaatcttgagcaccaaccacctaaagacaggcatccttttcagtgctatgctacaaatcgtcgcaatcgcttcagcctcgtctccgtctactcgagagactcttcatgctttcgctctagacttcggccaggctttccaactacttgacgaccttcgagacgatcacccggagacaggtaaagaccgcaacaaagacgctggaaagtccactctagtaaacagacttggtgcggacgctgctagacagaagcttcgagagcatatcgacagtgcagataagcatctcactttcgcatgtccacaaggcggtgccatcagacagttcatgcacttgtggttcggtcaccacttggctgactggtctccggtaatgaagatcgcttgatgggacttatgcttatcgattggtgcgcattggcccttgttgtattcatcggtctacctcacggagcacttgatgcggctatctcattctctatgatctcgtcggcaaagcgaatcgctcgtctagccggaatcctattgatctacctcctactagccactgcattcttccttatctggtaccagctgcctgctttctcgctactaatcttcctcctcatctcaatcatccacttcggcatggcagacttcaacgccagccctagtaagctaaagtggccccacatcatcgctcacggtggggtggtgacggtgtggcttccccttatccaaaagaacgaagtaacgaagcttttctctatccttactaacggtccgactccaatcctttgggacatcctattgatcttcttcttgtgctggtctatcggcgtttgtcttcacacttacgagacattgcgtagcaagcactacaacatcgcattcgagctaatcggtttgatcttcctagcgtggtacgctcctcctctcgttacattcgctacctacttctgcttcatccactctaggagacatttcagtttcgtatggaagcaattgcaacacatgtcaagtaagaagatgatgatcggaagtgcaatcatcctatcttgcacgtcttggctcatcggaggcggaatctacttcttcctaaacagcaagatgatcgctagtgaggctgctctacaaactgtcttcatcggactcgccgctctgaccgtgcctcatatgatcttgatcgatttcatcttccgtcctcattcttcgcggatcaagatcaagaactaatgagagacttgatcctcgtcggcggcggtctagccaacggtcttatcgcctggcgtttgaggcagcgctacccacagctaaacttgcttctgatcgaagctggagaacagcctggaggtaaccatacttggagtttccacgaggacgatttgacacctggacagcatgcttggttggcacctctagtggctcacgcatggccgggttatgaggtgcagttcccagacctccgacgacgtctggcaagaggatactactctatcacttcggagcgcttcgccgaagcactccaccaagcacttggagaaaacatctggctaaactgctcagtgtcagaggttctaccaaatagtgtaaggctcgctaacggggaagctctattagccggggccgttatcgacggtaggggtgtaacggcctcttccgccatgcaaactggttaccagcttttcctaggtcagcaatggaggttgacacagcctcacggccttactgttccgatcctaatggacgcaacggtagcacagcaacagggttacaggtttgtttatacactgccactatcggccgatacgctgctaatcgaggacactaggtacgccaacgttccacaacgtgacgataacgcattgcgtcagacggtgacggactacgcgcactcgaagggatggcagcttgctcagttggagagagaagagacaggctgtctgcctatcacacttgcaggggacatccaggcactatgggcagacgctccaggagtaccgagatccggaatgagggcgggtttgttccatccaactacaggttactcacttccactagccgtggctctagccgacgccatcgctgactcgccaaggctgggaagcgtcccactttaccagctcactaggcaatttgctgagcgccactggcgccgacaaggtttctttcgcctattgaaccgtatgcttttcctagctggacgagaggagaatcgctggcgtgtaatgcagcgtttctacggtcttccagagccaacggtcgagaggttctacgcaggaagacttagtctattcgataaggctaggatccttacaggcaagcctccggtaccactaggagaagcttggagagccgcactaaaccattttcctgacagaagagacaaggggtgatggcaggaggagtatgccgaactaacgtgcagctacctggaaaggtcgtggttatcactggggcgaacactgggatcggtaaggagacggcacgtgagctcgcgtcgcgtggggctcgtgtatacatcgcatgcagggacgtcctgaagggtgagtctgctgcatccgagatccgagtagacaccaagaactcgcaggtgctagtacgaaagctcgacttgagcgacactaagagtatcagggcgttcgctgaaggtttcctggcagaggagaagcaacttcacatcctgatcaataacgcaggtgttatgatgtgcccgtactcgaaaacagcggacggattcgagacccatctaggtgtaaaccaccttggacacttcttgctgacttacttgctactagagcgactcaaggtttctgcgccagcaagggtcgtcaacgtatcgtcggtagcgcaccatatcgggaagatcccatttcacgacctacagtcggagaagagatactcgcgaggtttcgcttactgccactcgaagctagctaacgttctcttcactcgggaattggctaagcgtttgcaagggacaggtgtaacgacatacgcggttcaccctggggttgtcaggtctgagcttgttagacattcgtctcttctttgtctactatggcgtctattcagtcctttcgtgaagacagctcgggagggggctcagactagcttgcactgtgctctggcggaagggctagaacctctatctggaaagtacttcagtgattgcaagcggacctgggtgagtccaagagcgagaaacaacaagactgctgagagattgtggaatgtcagctgcgagctacttggaatccgatgggagcttaccgccgcactcgagcatcaccatcaccatcactga

SEQ ID NO: 24 - DNA, 3720 bp, Artificial Sequence
  Description of Artificial Sequence Synthetic polynucleotide
atggttagtggttcaaaggctggcgtatctccacatagggaaatcgaggtcatgcgacaatcaatcgacgatcacctagcaggcctgttgcccgagactgacagccaggatatcgtctcgttggccatgcgtgagggagttatggcgcctgggaagagaattcggcctttactgatgttgctagctgcacgcgacctcagataccaaggtagtatgcccacattgctcgatctagcctgcgcagtcgagctgacacatactgctagcctaatgctagatgacatgccatgtatggataacgcagagctacgtcgcgggcaacccacaacccacaagaaattcggtgagtctgtagctatcctagcaagcgtcggtctgcttagtaaggcattcggtttaatcgctgccactggtgacctgccaggagagagacgggctcaagccgtaaacgagctaagtactgcagtaggagttcagggtctagtactagggcaattccgagacctaaacgacgctgccctcgatagaacaccagacgcaatcttgagcaccaaccacctaaagacaggcatccttttcagtgctatgctacaaatcgtcgcaatcgcttcagcctcgtctccgtctactcgagagactcttcatgctttcgctctagacttcggccaggctttccaactacttgacgaccttcgagacgatcacccggagacaggtaaagaccgcaacaaagacgctggaaagtccactctagtaaacagacttggtgcggacgctgctagacagaagcttcgagagcatatcgacagtgcagataagcatctcactttcgcatgtccacaaggcggtgccatcagacagttcatgcacttgtggttcggtcaccacttggctgactggtctccggtaatgaagatcgcttgatgggacttatgcttatcgattggtgcgcattggcccttgttgtattcatcggtctacctcacggagcacttgatgcggctatctcattctctatgatctcgtcggcaaagcgaatcgctcgtctagccggaatcctattgatctacctcctactagccactgcattcttccttatctggtaccagctgcctgctttctcgctactaatcttcctcctcatctcaatcatccacttcggcatggcagacttcaacgccagccctagtaagctaaagtggccccacatcatcgctcacggtggggtggtgacggtgtggcttccccttatccaaaagaacgaagtaacgaagcttttctctatccttactaacggtccgactccaatcctttgggacatcctattgatcttcttcttgtgctggtctatcggcgtttgtcttcacacttacgagacattgcgtagcaagcactacaacatcgcattcgagctaatcggtttgatcttcctagcgtggtacgctcctcctctcgttacattcgctacctacttctgcttcatccactctaggagacatttcagtttcgtatggaagcaattgcaacacatgtcaagtaagaagatgatgatcggaagtgcaatcatcctatcttgcacgtcttggctcatcggaggcggaatctacttcttcctaaacagcaagatgatcgctagtgaggctgctctacaaactgtcttcatcggactcgccgctctgaccgtgcctcatatgatcttgatcgatttcatcttccgtcctcattcttcgcggatcaagatcaagaactaatgagagacttgatcctcgtcggcggcggtctagccaacggtcttatcgcctggcgtttgaggcagcgctacccacagctaaacttgcttctgatcgaagctggagaacagcctggaggtaaccatacttggagtttccacgaggacgatttgacacctggacagcatgcttggttggcacctctagtggctcacgcatggccgggttatgaggtgcagttcccagacctccgacgacgtctggcaagaggatactactctatcacttcggagcgcttcgccgaagcactccaccaagcacttggagaaaacatctggctaaactgctcagtgtcagaggttctaccaaatagtgtaaggctcgctaacggggaagctctattagccggggccgttatcgacggtaggggtgtaacggcctcttccgccatgcaaactggttaccagcttttcctaggtcagcaatggaggttgacacagcctcacggccttactgttccgatcctaatggacgcaacggtagcacagcaacagggttacaggtttgtttatacactgccactatcggccgatacgctgctaatcgaggacactaggtacgccaacgttccacaacgtgacgataacgcattgcgtcagacggtgacggactacgcgcactcgaagggatggcagcttgctcagttggagagagaagagacaggctgtctgcctatcacacttgcaggggacatccaggcactatgggcagacgctccaggagtaccgagatccggaatgagggcgggtttgttccatccaactacaggttactcacttccactagccgtggctctagccgacgccatcgctgactcgccaaggctgggaagcgtcccactttaccagctcactaggcaatttgctgagcgccactggcgccgacaaggtttctttcgcctattgaaccgtatgcttttcctagctggacgagaggagaatcgctggcgtgtaatgcagcgtttctacggtcttccagagccaacggtcgagaggttctacgcaggaagacttagtctattcgataaggctaggatccttacaggcaagcctccggtaccactaggagaagcttggagagccgcactaaaccattttcctgacagaagagacaaggggtgatgactcacaaggctacagagatcctaacagggaaggttatgcaaaagagtgtcctaatcactggttgcagttctggtatcggcctagagtcggctctagaactcaagagacagggtttccacgtgctggcgggatgcaggaagccagatgacgtcgaacgcatgaacagcatgggtttcactggagtattgatcgaccttgacagccccgagagtgtcgacagagcagcagatgaggttatcgcacttacagacaactgtctatacggtatcttcaacaacgcaggtttcggaatgtacggacctctatcgactatcagtcgagcacagatggaacagcagttcagtgcgaacttcttcggcgcgcaccagttgacaatgagacttcttcccgctatgttgccgcacggggaaggtcgcatcgtaatgacgtcttctgtaatgggactaatctcgacgcctggtagaggtgcgtacgcagcttcgaagtacgccttggaggcatggagcgacgcgctgcgtatggaacttaggcactcggggatcaaggttagcctgatcgagcctgggccaatccgcactcgattcaccgacaacgttaaccaaactcaatcagacaagcccgtcgaaaaccccggtatcgcggcacgattcacgcttggacctgaggccgtagttgacaaggttcgtcacgcattcatcagtgagaagccgaagatgcgatacccagtaactctggtaacctgggctgttatggttctaaagcgcctgctgccgggaagggtaatggataagatcctacagggatga

SEQ ID NO: 25 - DNA, 3756 bp, Artificial Sequence
  Description of Artificial Sequence Synthetic polynucleotide
atggttagtggttcaaaggctggcgtatctccacatagggaaatcgaggtcatgcgacaatcaatcgacgatcacctagcaggcctgttgcccgagactgacagccaggatatcgtctcgttggccatgcgtgagggagttatggcgcctgggaagagaattcggcctttactgatgttgctagctgcacgcgacctcagataccaaggtagtatgcccacattgctcgatctagcctgcgcagtcgagctgacacatactgctagcctaatgctagatgacatgccatgtatggataacgcagagctacgtcgcgggcaacccacaacccacaagaaattcggtgagtctgtagctatcctagcaagcgtcggtctgcttagtaaggcattcggtttaatcgctgccactggtgacctgccaggagagagacgggctcaagccgtaaacgagctaagtactgcagtaggagttcagggtctagtactagggcaattccgagacctaaacgacgctgccctcgatagaacaccagacgcaatcttgagcaccaaccacctaaagacaggcatccttttcagtgctatgctacaaatcgtcgcaatcgcttcagcctcgtctccgtctactcgagagactcttcatgctttcgctctagacttcggccaggctttccaactacttgacgaccttcgagacgatcacccggagacaggtaaagaccgcaacaaagacgctggaaagtccactctagtaaacagacttggtgcggacgctgctagacagaagcttcgagagcatatcgacagtgcagataagcatctcactttcgcatgtccacaaggcggtgccatcagacagttcatgcacttgtggttcggtcaccacttggctgactggtctccggtaatgaagatcgcttgatgggacttatgcttatcgattggtgcgcattggcccttgttgtattcatcggtctacctcacggagcacttgatgcggctatctcattctctatgatctcgtcggcaaagcgaatcgctcgtctagccggaatcctattgatctacctcctactagccactgcattcttccttatctggtaccagctgcctgctttctcgctactaatcttcctcctcatctcaatcatccacttcggcatggcagacttcaacgccagccctagtaagctaaagtggccccacatcatcgctcacggtggggtggtgacggtgtggcttccccttatccaaaagaacgaagtaacgaagcttttctctatccttactaacggtccgactccaatcctttgggacatcctattgatcttcttcttgtgctggtctatcggcgtttgtcttcacacttacgagacattgcgtagcaagcactacaacatcgcattcgagctaatcggtttgatcttcctagcgtggtacgctcctcctctcgttacattcgctacctacttctgcttcatccactctaggagacatttcagtttcgtatggaagcaattgcaacacatgtcaagtaagaagatgatgatcggaagtgcaatcatcctatcttgcacgtcttggctcatcggaggcggaatctacttcttcctaaacagcaagatgatcgctagtgaggctgctctacaaactgtcttcatcggactcgccgctctgaccgtgcctcatatgatcttgatcgatttcatcttccgtcctcattcttcgcggatcaagatcaagaactaatgagagacttgatcctcgtcggcggcggtctagccaacggtcttatcgcctggcgtttgaggcagcgctacccacagctaaacttgcttctgatcgaagctggagaacagcctggaggtaaccatacttggagtttccacgaggacgatttgacacctggacagcatgcttggttggcacctctagtggctcacgcatggccgggttatgaggtgcagttcccagacctccgacgacgtctggcaagaggatactactctatcacttcggagcgcttcgccgaagcactccaccaagcacttggagaaaacatctggctaaactgctcagtgtcagaggttctaccaaatagtgtaaggctcgctaacggggaagctctattagccggggccgttatcgacggtaggggtgtaacggcctcttccgccatgcaaactggttaccagcttttcctaggtcagcaatggaggttgacacagcctcacggccttactgttccgatcctaatggacgcaacggtagcacagcaacagggttacaggtttgtttatacactgccactatcggccgatacgctgctaatcgaggacactaggtacgccaacgttccacaacgtgacgataacgcattgcgtcagacggtgacggactacgcgcactcgaagggatggcagcttgctcagttggagagagaagagacaggctgtctgcctatcacacttgcaggggacatccaggcactatgggcagacgctccaggagtaccgagatccggaatgagggcgggtttgttccatccaactacaggttactcacttccactagccgtggctctagccgacgccatcgctgactcgccaaggctgggaagcgtcccactttaccagctcactaggcaatttgctgagcgccactggcgccgacaaggtttctttcgcctattgaaccgtatgcttttcctagctggacgagaggagaatcgctggcgtgtaatgcagcgtttctacggtcttccagagccaacggtcgagaggttctacgcaggaagacttagtctattcgataaggctaggatccttacaggcaagcctccggtaccactaggagaagcttggagagccgcactaaaccattttcctgacagaagagacaaggggtgatgactcacaaggctacagagatcctaacagggaaggttatgcaaaagagtgtcctaatcactggttgcagttctggtatcggcctagagtcggctctagaactcaagagacagggtttccacgtgctggcgggatgcaggaagccagatgacgtcgaacgcatgaacagcatgggtttcactggagtattgatcgaccttgacagccccgagagtgtcgacagagcagcagatgaggttatcgcacttacagacaactgtctatacggtatcttcaacaacgcaggtttcggaatgtacggacctctatcgactatcagtcgagcacagatggaacagcagttcagtgcgaacttcttcggcgcgcaccagttgacaatgagacttcttcccgctatgttgccgcacggggaaggtcgcatcgtaatgacgtcttctgtaatgggactaatctcgacgcctggtagaggtgcgtacgcagcttcgaagtacgccttggaggcatggagcgacgcgctgcgtatggaacttaggcactcggggatcaaggttagcctgatcgagcctgggccaatccgcactcgattcaccgacaacgttaaccaaactcaatcagacaagcccgtcgaaaaccccggtatcgcggcacgattcacgcttggacctgaggccgtagttgacaaggttcgtcacgcattcatcagtgagaagccgaagatgcgatacccagtaactctggtaacctgggctgttatggttctaaagcgcctgctgccgggaagggtaatggataagatcctacagggacttaccgccgcactcgagcatcaccatcaccatcactga

SEQ ID NO: 26 - DNA, 3307 bp, Artificial Sequence
  Description of Artificial Sequence Synthetic polynucleotide
atgaagaaaacagttgtcatcggcgcaggcttcgggggacttgcattggctatcagactgcaggccgcgggaatccccacagtcctattggagcagagagacaagccaggagggcgcgcatacgtctggcatgaccagggattcacttttgatgcaggtccgacggtaatcacagatcctactgcccttgaggctcttttcacactagctggtagaagaatggaggactacgtacgtctgttgcctgttaaacccttctaccgtttgtgttgggaaagcggtaagacacttgactacgcaaacgactcggctgagctcgaggctcagatcacccagttcaacccaagagatgtagaaggatacagaaggttccttgcgtactctcaggctgtttttcaggaaggataccttcgactaggatctgttccgtttctatcttttcgagacatgctacgcgctgggccacagttgctaaagctacaggcttggcagtcggtctaccagtcagtttcgaggttcatcgaggatgaacacctcagacaggctttctctttccactcgttactggttggtggtaacccattcactacaagttcgatttacaccctaatccacgccctagagagagagtggggggtttggttccctgagggcggcactggtgccttggtgaacggcatggtcaagctattcacggaccttggcggggaaattgaacttaacgccagagtcgaagaactagtggtagcagataatcgagttagccaagtcaggctggccgacggacgcatcttcgatactgacgctgtcgcttcgaacgcagacgtagtgaacacttacaagaagcttctcggccatcatccggtcggacagaaaagagctgcggcactggaacgtaagtcaatgtcgaactctctattcgtgttgtacttcggtctgaaccaaccacactctcagttggcccaccataccatctgttttggtccacgctaccgagagctaatcgatgagatcttcacaggaagcgctcttgcagacgacttttccctatacttgcattccccatgcgttactgacccatccctagctccaccaggatgtgctagtttctacgtgctagctcctgtgccacacctgggaaacgctccgctggattgggctcaggaaggaccaaagctgcgcgacaggattttcgattacctagaagaacgttacatgccaggcttgcgatctcagttggtaacacagcgtatcttcaccccagcagacttccacgatacgctagacgctcacctagggagtgctttcagcatcgaaccgctgttgacacaaagcgcctggttccgaccacacaaccgagactctgacatcgctaacctgtacctcgtgggtgcgggaactcaccctggggccggcatccctggtgtagtcgcttctgccaaggcaacagcatcattgatgatcgaagacctgcaataatgagccaacctccgttactcgaccatgcaactcagactatggccaacggatctaagtcgttcgcaacagcagctaagctcttcgacccagctacacgtcgatcggtgctaatgttgtacacctggtgcagacattgtgatgacgtaatcgacgatcagacacacggtttcgcctcagaagccgctgccgaagaggaagctacacagcgactagccaggttgcgcacattgacccttgcagctttcgagggtgctgagatgcaagacccggcattcgcggcctttcaagaggttgccctgacccacggtatcactcctcgtatggccttggatcacctagacggcttcgccatggacgttgcgcaaaccaggtatgttacttttgaagataccctaaggtactgctatcacgtcgccggcgttgtgggtcttatgatggctcgtgtaatgggtgtgcgtgatgaacgtgtactagatagggcttgtgacctaggactagcattccagctgaccaacatcgcacgagacatcatcgacgatgcagcaatcgatcgctgctacctccccgcagagtggctgcaggacgccggactgaccccggagaactatgctgccagggagaaccgtgccgcactagctcgagtagctgagcgcctaatcgacgcagcagagccatactacatctcaagccaggcagggcttcatgacctgccacctcgttgcgcttgggccatcgctacagctcgctcggtctacagggagatcgggatcaaggtaaaggccgctggaggttcggcctgggacagacgtcagcataccagcaagggtgagaagatcgccatgctaatggccgcaccgggacaagttatccgtgcaaaaactacgcgtgttactccacggccagctggtctttggcaacggcctgtctaatggacttccctcagcaacttgaggcttgcgtcaagcaggcgaaccaggctttgtctaggttcatcgcgcctctacccttccagaacacacctgtagtagagaccatgcagtacggtgcactattgggtggaaagcgactaagacctttcttggtttacgctactggtcacatgttcggagtctcaacgaacacgctggatgcacctgcagctgcagtagagtgtatccacgcatactcgctaatccacgacgatctcccggctatggacgacgacgacctacgtcgcggattgcctacttgccacgtaaagttcggcgaggcaaacgcaatcctcgcaggtgacgctctccagacactggccttcagtatcttgtcggatgcggatatgccagaggtatctgatagagatcgaatctctatgatctcggagttggcatctgcgagcggaatcgcagggatgtgcggaggacaggctcttgatcttgacgctgagggtaagcatgttcccctggacgcactcgagaggatccacagacacaagactggtgcactcatccgagctgctgtccgtctcggagcgctcagcgctggggacaagggacgaagggcactgccagtactagacaagtacgcagagtctatcggactagctttccaggtacaggatgatatcctagatgttgttggtgacactgctactctaggtaagaggcagggggcagaccagcagttggggaagagtacttaccccgcacttctagggttggagcaggcgaggaagaaggcgagagacctgatcgacgatgctcgccagagtctaaagcaattggctgagcagtcgttggatacatctgctcttgaggcccttgccgactacatcatccaacgaaacaagtga

SEQ ID NO: 27 - DNA, 924 bp, Artificial Sequence
  Description of Artificial Sequence Synthetic polynucleotide
atggttagtggttcaaaggctggcgtatctccacatagggaaatcgaggtcatgcgacaatcaatcgacgatcacctagcaggcctgttgcccgagactgacagccaggatatcgtctcgttggccatgcgtgagggagttatggcgcctgggaagagaattcggcctttactgatgttgctagctgcacgcgacctcagataccaaggtagtatgcccacattgctcgatctagcctgcgcagtcgagctgacacatactgctagcctaatgctagatgacatgccatgtatggataacgcagagctacgtcgcgggcaacccacaacccacaagaaattcggtgagtctgtagctatcctagcaagcgtcggtctgcttagtaaggcattcggtttaatcgctgccactggtgacctgccaggagagagacgggctcaagccgtaaacgagctaagtactgcagtaggagttcagggtctagtactagggcaattccgagacctaaacgacgctgccctcgatagaacaccagacgcaatcttgagcaccaaccacctaaagacaggcatccttttcagtgctatgctacaaatcgtcgcaatcgcttcagcctcgtctccgtctactcgagagactcttcatgctttcgctctagacttcggccaggctttccaactacttgacgaccttcgagacgatcacccggagacaggtaaagaccgcaacaaagacgctggaaagtccactctagtaaacagacttggtgcggacgctgctagacagaagcttcgagagcatatcgacagtgcagataagcatctcactttcgcatgtccacaaggcggtgccatcagacagttcatgcacttgtggttcggtcaccacttggctgactggtctccggtaatgaagatcgcttga

SEQ ID NO: 28 - DNA, 1161 bp, Artificial Sequence
  Description of Artificial Sequence Synthetic polynucleotide
atgagagacttgatcctcgtcggcggcggtctagccaacggtcttatcgcctggcgtttgaggcagcgctacccacagctaaacttgcttctgatcgaagctggagaacagcctggaggtaaccatacttggagtttccacgaggacgatttgacacctggacagcatgcttggttggcacctctagtggctcacgcatggccgggttatgaggtgcagttcccagacctccgacgacgtctggcaagaggatactactctatcacttcggagcgcttcgccgaagcactccaccaagcacttggagaaaacatctggctaaactgctcagtgtcagaggttctaccaaatagtgtaaggctcgctaacggggaagctctattagccggggccgttatcgacggtaggggtgtaacggcctcttccgccatgcaaactggttaccagcttttcctaggtcagcaatggaggttgacacagcctcacggccttactgttccgatcctaatggacgcaacggtagcacagcaacagggttacaggtttgtttatacactgccactatcggccgatacgctgctaatcgaggacactaggtacgccaacgttccacaacgtgacgataacgcattgcgtcagacggtgacggactacgcgcactcgaagggatggcagcttgctcagttggagagagaagagacaggctgtctgcctatcacacttgcaggggacatccaggcactatgggcagacgctccaggagtaccgagatccggaatgagggcgggtttgttccatccaactacaggttactcacttccactagccgtggctctagccgacgccatcgctgactcgccaaggctgggaagcgtcccactttaccagctcactaggcaatttgctgagcgccactggcgccgacaaggtttctttcgcctattgaaccgtatgcttttcctagctggacgagaggagaatcgctggcgtgtaatgcagcgtttctacggtcttccagagccaacggtcgagaggttctacgcaggaagacttagtctattcgataaggctaggatccttacaggcaagcctccggtaccactaggagaagcttggagagccgcactaaaccattttcctgacagaagagacaaggggtga

SEQ ID NO: 29 - DNA, 1479 bp, Artificial Sequence
  Description of Artificial Sequence Synthetic polynucleotide
atgaagaaaacagttgtcatcggcgcaggcttcgggggacttgcattggctatcagactgcaggccgcgggaatccccacagtcctattggagcagagagacaagccaggagggcgcgcatacgtctggcatgaccagggattcacttttgatgcaggtccgacggtaatcacagatcctactgcccttgaggctcttttcacactagctggtagaagaatggaggactacgtacgtctgttgcctgttaaacccttctaccgtttgtgttgggaaagcggtaagacacttgactacgcaaacgactcggctgagctcgaggctcagatcacccagttcaacccaagagatgtagaaggatacagaaggttccttgcgtactctcaggctgtttttcaggaaggataccttcgactaggatctgttccgtttctatcttttcgagacatgctacgcgctgggccacagttgctaaagctacaggcttggcagtcggtctaccagtcagtttcgaggttcatcgaggatgaacacctcagacaggctttctctttccactcgttactggttggtggtaacccattcactacaagttcgatttacaccctaatccacgccctagagagagagtggggggtttggttccctgagggcggcactggtgccttggtgaacggcatggtcaagctattcacggaccttggcggggaaattgaacttaacgccagagtcgaagaactagtggtagcagataatcgagttagccaagtcaggctggccgacggacgcatcttcgatactgacgctgtcgcttcgaacgcagacgtagtgaacacttacaagaagcttctcggccatcatccggtcggacagaaaagagctgcggcactggaacgtaagtcaatgtcgaactctctattcgtgttgtacttcggtctgaaccaaccacactctcagttggcccaccataccatctgttttggtccacgctaccgagagctaatcgatgagatcttcacaggaagcgctcttgcagacgacttttccctatacttgcattccccatgcgttactgacccatccctagctccaccaggatgtgctagtttctacgtgctagctcctgtgccacacctgggaaacgctccgctggattgggctcaggaaggaccaaagctgcgcgacaggattttcgattacctagaagaacgttacatgccaggcttgcgatctcagttggtaacacagcgtatcttcaccccagcagacttccacgatacgctagacgctcacctagggagtgctttcagcatcgaaccgctgttgacacaaagcgcctggttccgaccacacaaccgagactctgacatcgctaacctgtacctcgtgggtgcgggaactcaccctggggccggcatccctggtgtagtcgcttctgccaaggcaacagcatcattgatgatcgaagacctgcaataa

SEQ ID NO: 30 - protein, 291 aa, Artificial Sequence
  Description of Artificial Sequence Synthetic polypeptide
MAGGVCRTNVQLPGKVVVITGANTGIGKETARELASRGARVYIACRDVLKGESAASEIRVDTKNSQVLVRKLDLSDTKSIRAFAEGFLAEEKQLHILINNAGVMMCPYSKTADGFETHLGVNHLGHFLLTYLLLERLKVSAPARVVNVSSVAHHIGKIPFHDLQSEKRYSRGFAYCHSKLANVLFTRELAKRLQGTGVTTYAVHPGVVRSELVRHSSLLCLLWRLFSPFVKTAREGAQTSLHCALAEGLEPLSGKYFSDCKRTWVSPRARNNKTAERLWNVSCELLGIRWE

SEQ ID NO: 31 - DNA, 37 bp, Artificial Sequence
  Description of Artificial Sequence Synthetic oligonucleotide
tggtgcaaaacctttcgcggtatggcatgatagcgcc

SEQ ID NO: 32 - DNA, 26 bp, Artificial Sequence
  Description of Artificial Sequence Synthetic oligonucleotide
tactagagaaagaggggaaatactag

SEQ ID NO: 33 - DNA, 53 bp, Artificial Sequence
  Description of Artificial Sequence Synthetic oligonucleotide
ccaattattgaaggcctccctaacggggggcctttttttgtttctggtctccc

SEQ ID NO: 34 - DNA, 31 bp, Artificial Sequence
  Description of Artificial Sequence Synthetic oligonucleotide
tttacactttatgcttccggctcgtatgttg

SEQ ID NO: 35 - DNA, 27 bp, Artificial Sequence
  Description of Artificial Sequence Synthetic oligonucleotide
tagtacattaaagaggagaaatagtac

SEQ ID NO: 36 - DNA, 76 bp, Artificial Sequence
  Description of Artificial Sequence Synthetic oligonucleotide
tttacactttatgcttccggctcgtatgttgtgtggaattgtgagcgtctagtagaaggaggagatctggatccat

SEQ ID NO: 37 - DNA, 61 bp, Artificial Sequence
  Description of Artificial Sequence Synthetic oligonucleotide
ctcggtaccaaattccagaaaagaggcctcccgaaaggggggccttttttcgttttggtcc

SEQ ID NO: 38 - DNA, 60 bp, Artificial Sequence
  Description of Artificial Sequence Synthetic oligonucleotide
aaaacctttcgcggtatggcatgatagcgcccggaagagagtcaattcagggaggtgaat

SEQ ID NO: 39 - protein, 6 aa, Artificial Sequence
  Description of Artificial Sequence Synthetic 6xHis tag
HHHHHH